(19) United States
(12) Patent Application Publication
(10) Pub. No.: US 2026/0134963 A1
(43) Pub. Date: May 14, 2026

(54) Fuzzy Neural Network Physiologic Closed Loop Control System for Diuretic Management

(71) Applicant: REGENTS OF THE UNIVERSITY OF MICHIGAN, Ann Arbor, MI (US)

(72) Inventors: Kayvan Najarian, Northville, MI (US); Matthew Hodgman, Wilmette, IL (US); Daniel Ehrmann, Ann Arbor, MI (US)

(21) Appl. No.: 19/371,551

(22) Filed: Oct. 28, 2025

Related U.S. Application Data
(60) Provisional application No. 63/719,511, filed on Nov. 12, 2024.

(51) Int. Cl. G16H 20/00; G06N 5/048

(52) U.S. Cl. CPC G16H 20/00; G06N 5/048

(57) ABSTRACT

Methods for controlling fluid management for a patient are disclosed herein. An example computer-implemented method comprises: initializing a fuzzy machine learning model based on a fluid management ruleset for a population including the patient; training the fuzzy machine learning model on historical medical data associated with the population to generate a trained fuzzy ruleset, the historical medical data including a plurality of indications of historical fluid management treatments for the population and associated treatment outcomes; receiving clinical data for the patient; generating a fluid management recommendation by applying the trained fuzzy ruleset to the clinical data for the patient; presenting, via a graphical user interface, the fluid management recommendation and one or more indications of corresponding rules included in the trained fuzzy ruleset to a clinician.

### CROSS-REFERENCE TO RELATED APPLICATION

The present application claims the priority benefit of U.S. Provisional Application No. 63/719,511, filed Nov. 12, 2024, the entire disclosure of which is hereby incorporated by reference.

### FIELD OF THE DISCLOSURE

The present disclosure generally relates to fuzzy machine learning models for controlling fluid management for a patient and, more particularly, to generating and training a fuzzy machine learning model and/or a fluid management ruleset for a fuzzy machine learning model for a patient.

### BACKGROUND

Machine learning (ML) and artificial intelligence (AI) models are used by professionals in a wide variety of fields to analyze large quantities of data and make determinations based on said data. To properly analyze the data and/or make determinations, the ML and AI models often rely on rules programmed into the models in question. However, high performance models that are capable of analyzing complicated problems are often difficult for a user to understand. Notably, such “black box” models lack transparency and justification of the output recommendations and/or analysis, especially for the average individual user, who is often not trained in ML and/or AI programming.

Moreover, present ML and AI models require precise annotated data for training, and cannot properly analyze or use the same approximate or “fuzzy” rules that human experts do. Many ML and AI models, for example, cannot properly determine that a parameter is “a little high” the same way a human expert would. As such, an interpretable model for machine learning and/or artificial intelligence with a mechanism to leverage approximate knowledge for formation or training of the model and/or rulesets is desired.

Fluid overload after congenital heart surgery is independently associated with significant morbidity and mortality. Managing fluid overload currently involves manual titration of diuretics, but this strategy is highly variable between providers and centers, and may cause unintentional under- or over-diuresis. Due to the complexity of fluid management and the high stakes involved, present ML and AI models are not suitable for controlling fluid management in a patient. Additionally, present ML and AI models are not equipped to provide interpretable models that allow clinicians to review and oversee an AI/ML based fluid management system.

### SUMMARY

In one embodiment, a computer-implemented method for controlling fluid management for a patient may be provided. The computer-implemented method may be implemented via one or more local or remote processors, servers, sensors, transceivers, memory units, and/or other electronic or electrical components. The computer-implemented method may include: initializing, by one or more processors, a fuzzy machine learning model based on a fluid management ruleset for a population including the patient; training, by the one or more processors, the fuzzy machine learning model on historical medical data associated with the population to generate a trained fuzzy ruleset, the historical medical data including a plurality of indications of historical fluid management treatments for the population and associated treatment outcomes; receiving, by the one or more processors, clinical data for the patient; generating, by the one or more processors, a fluid management recommendation by applying the trained fuzzy ruleset to the clinical data for the patient; presenting, by the one or more processors and via a graphical user interface, the fluid management recommendation and one or more first indications of corresponding rules included in the trained fuzzy ruleset to a clinician; receiving, by the one or more processors and via the graphical user interface, feedback data input by a clinician including an adjustment to at least one rule of the trained fuzzy ruleset; generating, by the one or more processors, an adjusted fluid management recommendation by applying an updated fuzzy ruleset to the clinical data; and presenting, by the one or more processors and via the graphical user interface, the adjusted fluid management recommendation and one or more second indications of corresponding rules included in the trained fuzzy ruleset to the clinician. It should be understood that the patient population may include neonatal, pediatric, and/or adult populations without limitation to a particular age group or clinical context.

In a variation of this embodiment, the population includes neonatal patients after congenital heart surgery.

In a variation of this embodiment, the computer-implemented method further comprises: obtaining, by one or more processors, clinician expertise data associated with fluid management procedure for the population; encoding, by the one or more processors, the clinician expertise data into fuzzy concepts; and generating, by the one or more processors, the fluid management ruleset based on the fuzzy concepts . . .

In a variation of this embodiment, the generating the trained fuzzy ruleset comprises: training, by the one or more processors and using the fuzzy machine learning model, the fluid management ruleset based on the clinician expertise data by: approximating, using tropical geometry, a continuous representation of the piecewise categorizing function, and generating, based on at least the continuous representation of the piecewise categorizing function, the trained fuzzy ruleset.

In a variation of this embodiment, the fluid management recommendation is a diuretics dosage recommendation or a fluid dosage recommendation.

In a variation of this embodiment, the computer-implemented method further comprises: administering, by the one or more processors and via a medical pump device, a diuretics dosage or a fluid dosage to the patient based on the fluid management recommendation or the adjusted fluid management recommendation. For example, the computer-implemented method may operate in advisory, semi-autonomous (e.g., with clinician confirmation), or autonomous modes. Clinician feedback may update rules and subsequent recommendations.

In a variation of this embodiment, the historical medical data further includes electronic health records (EHR) data for a plurality of patients in the population and historical clinical data for a plurality of patients in the population.

In a variation of this embodiment, the computer-implemented method further comprises: obtaining, by the one or more processors, clinical data for the patient on a continuous basis, wherein fluid management recommendations are generated based on the most recent clinical data and on a periodic basis. Recommendations may also be generated event-driven (e.g., upon threshold crossings, receipt of new data, or clinician request).

In a variation of this embodiment, receiving the feedback data comprises: generating, by the one or more processors, an alert including an indication of a request for the clinician to provide an adjustment to the fluid management recommendation; and presenting, via the graphical user interface, the alert to the clinician. Alerts may include awareness notifications when context-specific criteria are met and requests for review prior to action. As described above, a variation of this embodiment may operate in advisory, semi-autonomous, or autonomous modes where clinician feedback may update rules and subsequent recommendations.

In a variation of this embodiment, the clinical data for the patient include one or more of: fluid balance data, respiratory data, oxygen saturation data, heart rate data, blood pressure data, or body temperature data. Clinical data may include, for example, laboratory values (e.g., creatinine, electrolytes, blood urea nitrogen, etc.), device-derived parameters (e.g., ventilator settings, oxygenation parameters, hemodynamic metrics, etc.), and clinician-entered targets/goals in addition to the enumerated vital signs.

In another embodiment, a system for controlling fluid management for a patient may be provided. The system may include: one or more medical devices including at least a medical monitoring device and a medical pump device; one or more processors; one or more memories including computer-executable instructions that, when executed by the one or more processors, cause the computing system to: initialize a fuzzy machine learning model based on a fluid management ruleset for a population including the patient; train the fuzzy machine learning model on historical medical data associated with the population to generate a trained fuzzy ruleset, the historical medical data including a plurality of indications of historical fluid management treatments for the population and associated treatment outcomes; receive, from the medical monitoring device, clinical data for the patient; generate a fluid management recommendation by applying the trained fuzzy ruleset to the clinical data for the patient; presenting, via a graphical user interface, the fluid management recommendation and one or more first indications of corresponding rules included in the trained fuzzy ruleset to a clinician; receiving, via the graphical user interface, feedback data input by a clinician including an adjustment to at least one rule of the trained fuzzy ruleset; generate an adjusted fluid management recommendation by applying an updated fuzzy ruleset to the clinical data; and present, by the one or more processors and via the graphical user interface, the adjusted fluid management recommendation and one or more second indications of corresponding rules included in the trained fuzzy ruleset to the clinician. As described above, the patient population may include neonatal, pediatric, and/or adult populations without limitation to a particular age group or clinical context.

In a variation of this embodiment, the population includes neonatal patients after congenital heart surgery.

In a variation of this embodiment, the computer-executable instructions, when executed by the one or more processors, further cause the computing system to: obtain clinician expertise data associated with fluid management procedure for the population; encode the clinician expertise data into fuzzy concepts; and generate the fluid management ruleset based on the fuzzy concepts.

In a variation of this embodiment, the computer-executable instructions, when executed by the one or more processors, generate the trained fuzzy ruleset by causing the computing system to: train, using the fuzzy machine learning model, the fluid management ruleset based on the clinician expertise data by: approximating, using tropical geometry, a continuous representation of the piecewise categorizing function, and generating, based on at least the continuous representation of the piecewise categorizing function, the trained fuzzy ruleset.

In a variation of this embodiment, the fluid management recommendation is a diuretics dosage recommendation or a fluid dosage recommendation.

In a variation of this embodiment, the computer-executable instructions, when executed by the one or more processors, further cause the computing system to: administer, via the medical pump device, a diuretics dosage or a fluid dosage to the patient based on the fluid management recommendation or the adjusted fluid management recommendation.

In a variation of this embodiment, the historical medical data further includes electronic health records (EHR) data for a plurality of patients in the population and historical clinical data for a plurality of patients in the population.

In a variation of this embodiment, the computer-executable instructions, when executed by the one or more processors, further cause the computing system to: obtain clinical data for the patient on a continuous basis, wherein fluid management recommendations are generated based on the most recent clinical data and on a periodic basis.

In a variation of this embodiment, the computer-executable instructions, when executed by the one or more processors, receive the feedback data by causing the computing system to: generate, by the one or more processors, an alert including an indication of a request for the clinician to provide an adjustment to the fluid management recommendation; and present, via the graphical user interface, the alert to the clinician.

In a variation of this embodiment, the clinical data for the patient include one or more of: fluid balance data, respiratory data, oxygen saturation data, heart rate data, blood pressure data, or body temperature data.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 is a block diagram of an example logic circuit for implementing example methods and/or operations for controlling fluid management for a patient, in accordance with some embodiments described herein.

FIG. 2 illustrates a block diagram depicting an example application architecture for training and/or applying a model using a tropical geometry-based interpretable machine learning method, in accordance with some embodiments described herein.

FIG. 3A illustrates a diagram depicting methods for receiving an input dataset and extracting rules therefrom as well as for receiving a trained model and dataset and subsequently developing a summarized ruleset, implemented by the application of FIG. 2, in accordance with some embodiments described herein.

FIG. 3B illustrates a diagram depicting further systems and methods for receiving an input dataset and extracting rules therefrom, implemented by the application of FIG. 2, in accordance with some embodiments described herein.

FIG. 4 illustrates an example visualization of determined membership functions for concepts of various input variables involved in a rule of a ruleset, in accordance with some embodiments described herein.

FIG. 5A illustrates an example visualization of rulesets and concepts, as well as contributions of the rulesets to positive classes, as may be presented via an interactive user interface of a computing device, in accordance with some embodiments described herein.

FIG. 5B illustrates an example visualization of rulesets and concepts, as well as contributions of the rulesets to positive classes, as may be presented via an interactive user interface of a computing device, in accordance with some embodiments described herein.

FIG. 6A illustrates an example visualization of rulesets and concepts, as well as contributions of the rulesets to positive classes, as may be presented via an interactive user interface of a computing device, in accordance with some embodiments described herein.

FIG. 6B illustrates an example visualization of rulesets and concepts, as well as contributions of the rulesets to positive classes, as may be presented via an interactive user interface of a computing device, in accordance with some embodiments described herein.

FIG. 7 illustrates an example visualization of predicted and actual diuretics dose changes for a test set, in accordance with some embodiments described herein.

FIG. 8 illustrates an example visualization of various models used to inform a closed loop control system for fluid management of an example patient, in accordance with some embodiments described herein.

FIG. 9 illustrates a flowchart depicting an exemplary method for controlling fluid management for a patient, implemented in the computing environment of FIG. 1, in accordance with some embodiments described herein.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

When evaluating or describing concepts and/or characteristics, human actors use fuzzy descriptions without a strict definition. Such practices are true even for experts making difficult and/or complicated evaluations. For example, a doctor evaluating a patient heart rate may note that the heart rate is “a little high” without explicitly defining the boundaries of what heart rate range constitutes “a little high”. Another expert or, depending on the situation, a layperson may roughly understand the evaluation despite the lack of hard numbers. Similarly, a stock broker may describe a particular stock as “low performing” or a network analyst may describe security as “light”, and another evaluator would have a rough idea as to what the expert means despite the lack of precision.

However, computers using standard analysis techniques cannot understand the intuitive fuzzy concepts that human experts may understand naturally, leading to disconnects between the computers and the human experts using the computers. Implementing fuzzy concepts into computer-understandable techniques leads to algorithms and functions that are slow or inaccurate. In particular, using piecewise functions to directly represent the fuzzy concepts that are more intuitive to humans provides better accuracy. However, such functions cannot be analyzed and/or improved using standard techniques, such as gradient-based techniques, which are only usable for differentiable functions, but guarantee a convergence. For example, Gaussian functions are a common class of differentiable functions utilized in place of such piecewise functions for gradient-based techniques. The use of such Gaussian functions, however, loses accuracy. Similarly, available techniques for sharper, piecewise functions, such as genetic algorithms, also lack in speed and/or accuracy. Combinatorial or exhaustive search techniques, for example, are often implemented for encoding input data for such functions and require long periods of time to approach any sustainable level of accuracy. Similarly, techniques besides gradient techniques may not guarantee convergence, or may take prohibitive quantities of time to do so.

By using tropical geometry, a system may approximate the sharp piecewise functions with smooth continuous approximations. In some implementations, the system may approximate the smooth continuous approximations such that the smooth approximations are infinitely close to the sharp edges while still being continuous. Similarly, the system may take data from any number of sources to clarify how to encode various fuzzy concepts and how to determine which concepts and/or variables are relevant to a final determination.

By using tropical geometry to approximate fuzzy concepts, a system can generate and/or pull rules from a model and train the rules to improve the general accuracy and speed of making determinations. Similarly, because the concepts are fuzzy, the model is transparent and easy for humans to understand, unlike standard techniques, in which the determinations are largely oblique and/or difficult for even experts to understand.

Further, a system using tropical geometry to approximate fuzzy concepts can (i) improve good rules using input data, (ii) create rules from only input data, or (iii) determine that a ruleset is incomplete and/or incorrect. As such, the system is capable of removing irrelevant rules or generating new rules to improve the overall determination made by an expert or individual assessing or evaluating a condition. Similarly, such a system may present combinations of rules and/or variables that would normally be overlooked or missed by traditional systems that rely on receiving rules to train. Moreover, incorporating good rules into a model reduces the amount of training data needed to achieve a given level of accuracy in performance, generally improving the overall system performance.

Moreover, because the techniques described herein are able to determine and remove redundant, unimportant, or incorrect rules and/or data, the system may make determinations as to improved rules and/or variables agnostic to the inputs, whether the input rules are accurate, and/or the type of data the system receives. For example, a model created by the techniques described herein may receive numerical data, binary data, ordinal data, continuous data, categorical data, or any other similar data type as input and use the different types in conjunction. As such, the system can handle noise that would otherwise destroy any convergence that standard techniques could potentially attain.

Generally, the techniques described herein may be for implementing a closed loop control systems (CLCS) that adjust variables in a system to maintain a desired steady state, similar to cruise control in cars. The techniques may operate in advisory, semi-autonomous (e.g., with clinician confirmation), or autonomous modes. In each mode clinician feedback may update rules and subsequent recommendations, for example. Moreover, the techniques disclosed herein generally relate to a CLCS that automatically titrates a furosemide infusion based on clinical information and a desired fluid balance. The CLCS of the present invention includes one or more models developed using and/or including a tropical geometry-based fuzzy neural network (TGFNN) that function as a fluid management controller.

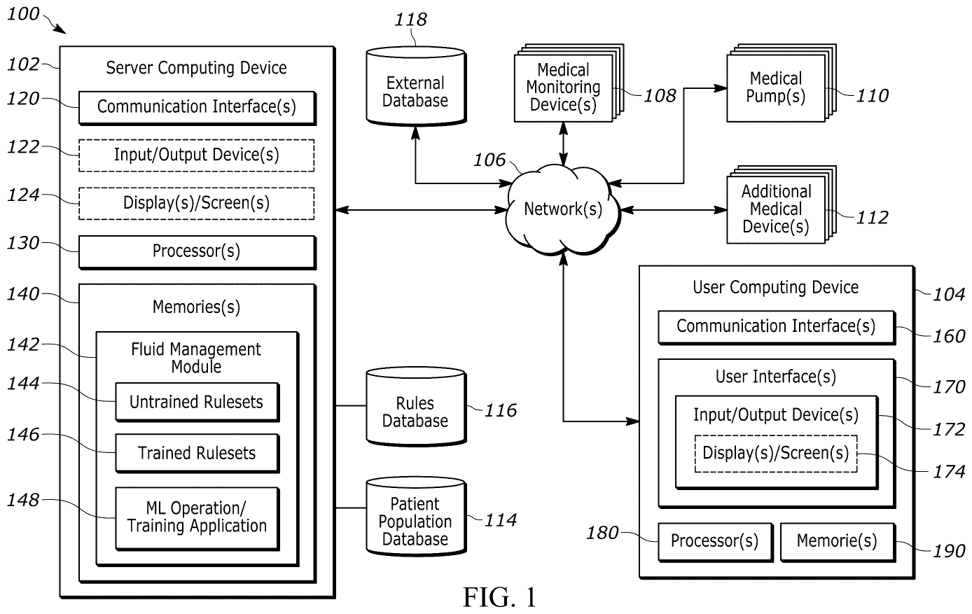

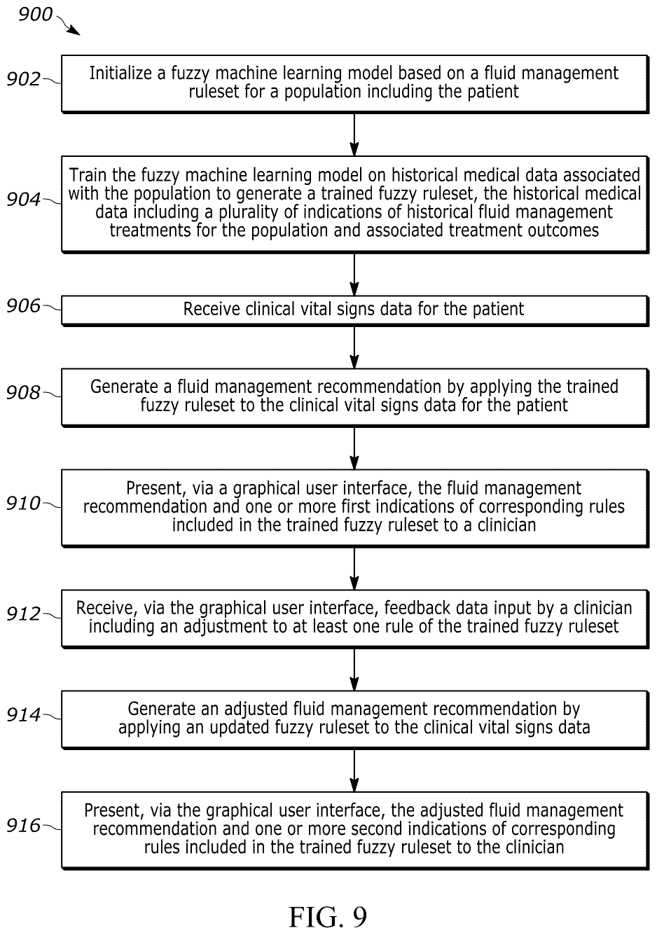

Referring now to the drawings, FIG. 1 is a block diagram representative of an example computing environment 100 capable of implementing the example methods and/or operations described herein, including, for example, one or more steps of the method 900 of FIG. 9 discussed in greater detail below. In some embodiments, the computing environment may be, or may be associated with, a medical environment. The computing environment 100 of FIG. 1 includes a server computing device 102, a user computing device 104, and one or more networks 106. In some embodiments, the computing environment 100 further includes one or more medical monitoring devices 108, one or more medical pumps 110, and/or one or more additional medical devices 112. Additionally or alternatively, the computing environment 100 may further include a patient population database 114, a rules database 116, and/or an external database 118. The exemplary network 106 of FIG. 1 may be a single communication link directly connecting the server computing device 102 and the user computing device 104 (e.g., a direct wired/wireless link), or one or more networks 106 may include multiple links (e.g., connecting the server computing device 102, the user computing device 104, the medical monitoring device 108, the medical pump 110, the additional medical device 112, the patient population database 114, the rules database 116, the external database 118, and/or an additional computing device) and/or communication networks of one or more types (e.g., one or more wired and/or wireless local area networks (LANs), and/or one or more wired and/or wireless wide area networks (WANs) such as the Internet, public networks, private networks, etc.). The network 106 may include any number of network devices, such as gateways, switches, routers, modems, repeaters, and wireless access points, among others. Additionally or alternatively, the network 106 may also include computing devices such as computer servers. For ease of reading herein (and not for limitation purposes), the one or more networks 106 may be referred to using the singular tense.

The example medical monitoring devices 108 may detect clinical data for a patient, including vital signs such as respiratory data, oxygen saturation data (SPO2), heart rate data, blood pressure data, body temperature data, etc. For example, the clinical data detected by the medical monitoring devices 108 may include fluid balance data (e.g., fluid balance including all fluid inputs and outputs), vital signs, raw or derived physiological monitoring data (e.g., near infrared spectroscopy, cardiac index, etc.), ventilator and/or respiratory parameters (e.g., type and amount of respiratory support), laboratory data, imaging data, and/or other clinical data from electronic health records and/or other clinical databases (e.g., the patient population database 114. Further, the one or more medical monitoring devices 108 may be fluid balance monitors, heart rate monitors, blood pressure monitors, thermometers, and/or other medical devices suitable for detecting clinical data for a patient. For ease of reading herein (and not for limitation purposes), the one or more medical monitoring devices 108 may be referred to using the singular tense. The medical monitoring devices 108 may be communicatively connected to the respective components of the computing environment 100, such as the server computing device 102, and may be configured to transmit the clinical data to one or more of the respective components. In some embodiments, the medical monitoring devices 108 may be configured to receive control and/or command instructions from the respective component of the computing environment 100 (e.g., to operate in advisory, semi-autonomous, or autonomous mode). In some embodiments, a medical monitoring device 108 may collect and/or obtain clinical data for a patient on a continuous basis (e.g., in real time while a patient is undergoing fluid management), and a fluid management recommendation may generated based on the most recent clinical data on a continuous basis and/or a periodic basis.

The example medical pumps 110 may deliver fluids, medication, blood products/derivatives, and/or nutrients to a patient. For example, the medical pump 110 may be intravenous (IV) pumps, syringe pumps, blood pumps, etc. As another example, the medical pump 110 may be configured to deliver a diuretics medication, such as a dosage of furosemide, to a patient. For ease of reading herein (and not for limitation purposes), the one or more medical pumps 110 may be referred to using the singular tense. The medical pumps 110 may be communicatively connected to the respective components of the computing environment 100, such as the server computing device 102, and may be configured to receive control and/or command instruction from one or more of the respective components (e.g., to operate in advisory, semi-autonomous, or autonomous mode). Typically, the server computing device 102 and/or the user computing device 104 may send control and/or command instructions to the medical pump 110 based on a fluid management recommendation generated using a trained fuzzy ruleset (e.g., trained rulesets 146) or model from the ML operation/training application 148.

The example additional medical devices 112 may be any medical devices suitable for fluid analysis and/or fluid management. In some embodiments, the additional medical devices 112 may be an additional medical monitoring device 112 (e.g., similar to the medical monitoring device 108) and/or an additional medical pump 112 (e.g., similar to the medical pump 110), and the additional medical monitoring devices 112 may detect clinical data for a patient and/or perform functions in response to a fluid management recommendation. For example, the additional medical devices 112 may be medical monitoring devices, medical devices for automatic urine output measurements, fluid warmers, oxygenators and/or ventilators, renal replacement therapy devices, mechanical ventilators, nitric-oxide consoles, extracorporeal support systems, infusion pumps for vasoactive agents, etc. In some embodiments, data collected/obtained by the additional medical devices 112 may be used as inputs to the ML operation/training application 148, and/or the additional medical devices 112 may perform additional functions in response to the outputs of the fluid management module 142. For example, the fluid management module 142 may transmit a fluid management recommendation, or information associated with a fluid management recommendation (e.g., a set of control or command instructions), to the additional medical devices 112 that causes the additional medical devices 112 to perform a specific function. It should be understood that the fluid management module 142 may transmit recommendation data and associated metadata to multiple endpoints (e.g., user computing device 104, medical pump 110, external database 118 such as an EHR). For ease of reading herein (and not for limitation purposes), the one or more medical devices 112 may be referred to using the singular tense. The additional medical devices 112 may be communicatively connected to the respective components of the computing environment 100, such as the server computing device 102, and may be configured to receive control and/or command instruction from one or more of the respective components.

The server computing device 102 may be an individual server, a group (e.g., cluster) of multiple servers, a computing device (e.g., a personal computer, a laptop, a smart phone, a tablet, a wearable device, etc.), or another suitable type of computing device or system (e.g., a collection of computing resources). The server computing device 102 may include one or more communication interfaces 120, one or more processors 130, and one or more memories 140. In some embodiments, the server computing device 102 further includes one or more input/output devices 122 and/or one or more displays/screens 124. Further, the memories 140 may have stored thereon one or more modules (e.g., one or more sets of instructions) including at least a fluid management module 142.

The one or more communication interfaces 120 may enable communication with other machines (e.g., components of the computing environment 100 such as user computing device 104, medical monitoring device 108, medical pump 110, additional medical device 112, patient population database 114, rules database 116, external database 118, etc.) via, for example, the one or more networks 106. The example communication interface 120 may include any suitable type of communication interface(s) (e.g., wired and/or wireless interfaces) configured to operate in accordance with any suitable protocol(s). For example, the communication interfaces 120 may be configured to transmit and receive data using a suitable wired communication protocol such as an Ethernet protocol, a USB protocol, a RS-232/UART protocol, an I²C protocol, a SPI protocol, or wireless communication protocols such as a Bluetooth (e.g., Bluetooth Low Energy) protocol, a Wi-Fi® (IEEE 802.11 standard) protocol, a near-field communication (NFC) protocol, a cellular (e.g., GSM, CDMA, LTE, WiMAX, etc.) protocol, a peer-to-peer wireless protocol, a short-range wireless protocol, and/or other suitable wired or wireless communication protocols. In some embodiments, the communication interface 120 may utilize a combination of such protocols to improve data throughput and/or efficiency. In some embodiments, the communication interface 120 may be a network interface controller (NIC) and may include any suitable NIC, such as wired/wireless controllers (e.g., Ethernet controllers), and facilitate bidirectional/multiplexed networking over the network 106 between the server computing device 102 and the user computing device 104 and/or other components of the environment 100 (e.g., medical monitoring device 108, medical pump 110, additional medical device 112, patient population database 114, rules database 116, external database 118, etc.). In some embodiments, the communication interface 120 may include advanced features such as hardware acceleration, specialized networking protocols, device-specific command sets, etc.

The one or more input/output (I/O) devices 122 and/or the one or more displays/screens 124 of the server computing device 102 may enable receipt of user input and communication of output data to the user. Further, the I/O devices 122 and/or the displays/screens 124 (e.g., I/O devices similar to the I/O devices 172 and displays/screens similar to the displays/screens 174, as described below) may present interactive graphical user interfaces (GUIs) to a user. For example, the I/O devices 122 and/or the displays/screens 124 may present various information in connection with the execution of instructions stored on the memories 140 for a user to view and/or otherwise perceive. Similarly, the I/O devices 122 and/or the displays/screens 124 may receive manual adjustments, selections, and/or data, and/or may allow a user to interact in any of a variety of manners with the processors 130 during execution of the instructions stored on the memories 140.

The processors 130 may include one or more microprocessors, controllers, and/or any suitable type of processor, and the memories 140 (e.g., volatile memory, non-volatile memory, non-transitory computer-readable media, etc.) may be accessible by the processor 130 (e.g., via a memory controller). For example, the one or more processors 130 may include one or more central processing units, one or more graphics processing units, one or more field-programmable gate arrays, one or more application-specific integrated circuits, one or more tensor processing units, one or more digital signal processors, one or more neural processing units, one or more RISC-V processors, one or more coprocessors, one or more specialized processors/accelerators for artificial intelligence or machine learning-specific applications, one or more microcontrollers, etc. The processor 130 may interact with the memory 140 to obtain, for example, machine-readable instructions and/or computer-executable instructions stored in the memory 140 corresponding to, for example, the operations represented by the flowcharts of this disclosure (e.g., the method 900 of FIG. 9). For example, the memories 140 may include one or more random access memories, one or more read-only memories, one or more cache memories, one or more hard disk drives, one or more solid-state drives, one or more non-volatile memory express, one or more optical drives, one or more universal serial bus flash drives, one or more external hard drives, one or more network-attached storage devices, one or more cloud storage instances, one or more tape drives, etc. Additionally, the processors 130 may be communicatively coupled to and/or control the communication interface 120 to transmit and/or receive various information pursuant to executing of the instructions stored on the memories 140 via, for example, the network 106.

As noted above, the memories 140 may have stored thereon a fluid management module 142, for example, as one or more sets of computer-executable instructions. The fluid management module 142 includes one or more untrained fluid management rulesets 144, one or more trained fluid management rulesets 146, and a machine learning (ML) operation and training application 148. In some aspects, the memories 140 may have stored thereon one or more additional modules (not depicted). For example, the memories 140 may include additional storage, such as one or more operating systems (e.g., Microsoft Windows, GNU/Linux, Mac OSX, etc.). The operating systems may be configured to run the modules stored on the memories 140 (e.g., the fluid management module 142) during operation of the server computing device 102. The modules stored on the memories 140 (e.g., the fluid management module 142) may be implemented using any suitable computer programming language(s) (e.g., Python, JavaScript, C, C++, Rust, C#, Swift, Java, Go, LISP, Ruby, Fortran, etc.). Further, the modules stored on the memories 140 may be configured to communicate with one another (e.g., via inter-process communication, via a bus, via message queues, etc.). In some embodiments, the modules stored on the memories 140 may respond to network requests (e.g., via an application programming interface) or other requests received via the network 106 (e.g., via the user computing device 104 or other components of the environment 100). In some implementations, the memories 140 may store data structures and/or information related to, for example, the modules (e.g., the fluid management module 142 and/or the ML operation and training application 148) and/or algorithms used in training, testing, or utilizing models to create, extract, and/or improve rulesets according to analyzed input data and/or rulesets as described in more detail below.

As mentioned above, the fluid management module 142 stored on the memories 140 includes one or more untrained fluid management rulesets 144 and one or more trained fluid management rulesets 146. Typically, an example patient may be associated with one or more populations of patients (e.g., the patient is a neonatal patient that has undergone congenital heart surgery, and the population is neonatal patients that have undergone congenital heart surgery), and further, fluid management procedures administered by clinicians may differ between populations of patients. In some embodiments, the ML operation/training application 148 and/or the fluid management module 142 may generate the untrained fluid management rulesets 144 by encoding clinician expertise data associated with a fluid management procedure for a population into fuzzy concepts. In some embodiments, the ML operation/training application 148 may generate the trained rulesets 146 by training the untrained rulesets 144 on, for example, historical clinical data. Generally speaking, the trained rulesets 146 may encode fluid management procedure(s) specific to a population of patients and the fluid management module 142 may use the trained rulesets 146 to control fluid management for a patient of the corresponding population of patients.

The rules database 116 and/or the external database 118 can include servers or other computing devices to provide input data and/or input rules for the ML operation/training application 148. The input data and/or input rules can include raw data, such as electrocardiogram (ECG) signals; processed data, such as survey data; fuzzy rules, such as a rule stating that if ejection fraction (EF) is low, and peak oxygen consumption (pVO2) is low, then evaluate for heart transplant and/or mechanical circulatory support (HT/MCS); or any other similarly desired input data. In further implementations, the rules database 116 and/or external database 118 can receive and store data such as improved and/or new rulesets from the ML operation/training application 148. As mentioned above, a patient may be associated with one or more populations of patients. In some embodiments, the external database 118 may store clinician expertise data associated with fluid management procedures for a population of patients. In some embodiments, the rules database 116 and/or the external database 118 may store the untrained fluid management rulesets 144 generated based on clinician expertise data associated with fluid management procedure for a population of patients including the patient. Additionally or alternatively, the rules database 116 and/or the external database 118 may store the trained fluid management rulesets 146. Although FIG. 1 shows the rules database 116 as separate from the external database 118, the computing environment can include both databases 116 and 118 as a single combined database as well.

The patient population database 114 of the computing environment 100 may store historical medical data associated with one or more populations. For example, the patient population database 114 may store historical medical data associated with a population of neonatal patients that have undergone congenital heart surgery. The historical medical data for a population stored on the patient population database 114 may include electronic health records (EHR) for a plurality of patients in the population and/or historical clinical data (e.g., fluid balance data, respiratory data, oxygen saturation data, heart rate data, blood pressure data, body temperature data, etc.) for a plurality of patients in the population. For example, the EHR data may include medical history data (e.g., past illnesses, past surgeries, family medical history, etc.), medication data, biographic information (e.g., gender, height, weight, age), medical conditions, etc. As another example, the historical clinical data may include vital signs for a patient over a period of time (e.g., vital signs data for the duration of a patient's recovery from a surgery, fluid balance data and/or other vital signs data collected during fluid management for a patient, and/or other clinical data collected over other periods of time).

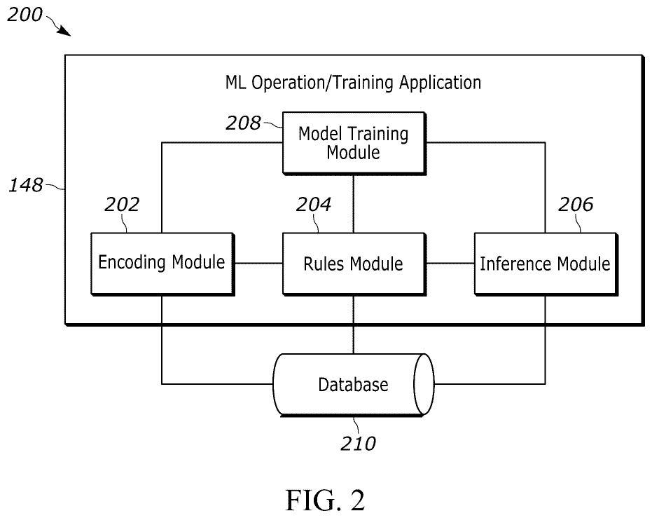

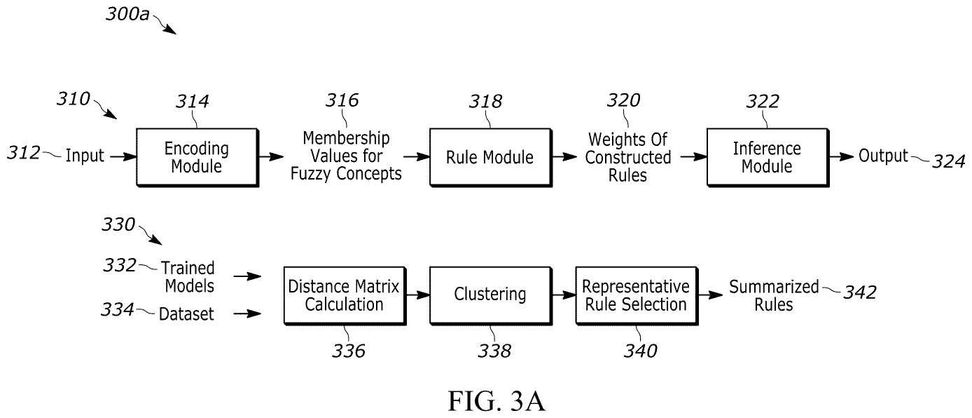

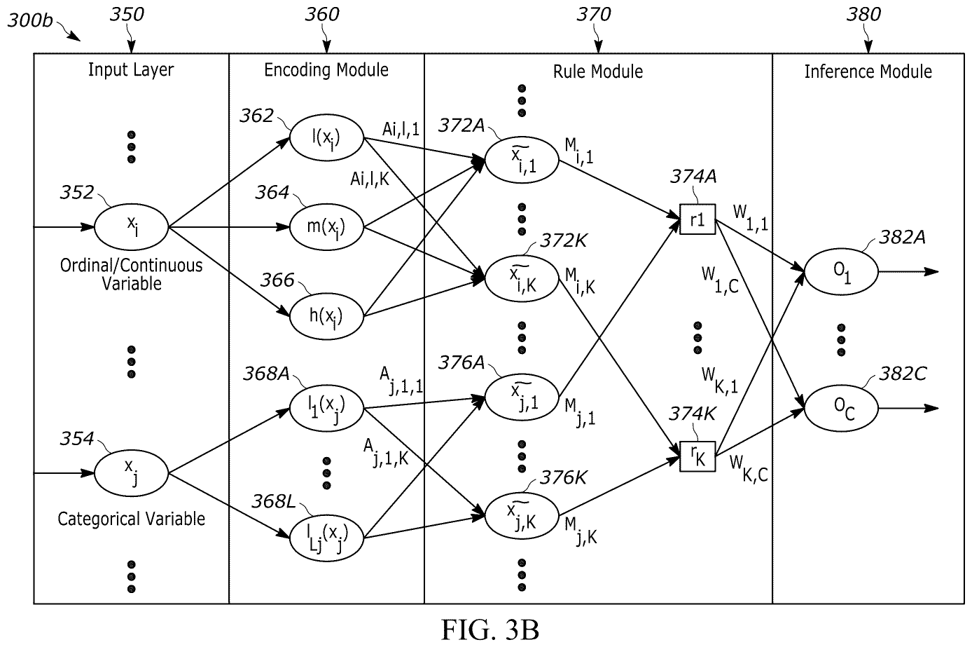

The memories 140 of the server computing device 102 of FIG. 1 may store instructions for executing a machine learning (ML) operation and training application 148. Generally speaking, the ML operation/training application 148 may include instructions for operating and/or training one or more machine learning models (e.g., a fuzzy machine learning model) and/or instructions for performing one or more of the operations described herein with respect to generating trained fuzzy rulesets. For example, the ML operation/training application 148 may include instructions for initializing a fuzzy machine learning model based on a fluid management ruleset (e.g., untrained rulesets 144, a rule or ruleset from the rules database 116). As another example, the ML operation/training application 148 may include instructions for training a fuzzy machine learning model to generate a trained fuzzy ruleset (e.g., the trained rulesets 146). Further, the ML operation/training application 148 may include instructions for training a fuzzy machine learning model on historical data (e.g., historical medical data associated with a population of patients, such as indications of historical fluid management treatments for a population and associated treatment outcomes; historical medical data from the patient population database 114) to generate a trained fuzzy ruleset. For example, the ML operation/training application 148 may retrieve data and/or information (e.g., historical medical data, rules and/or rulesets, etc.) for training or implementing a model from the patient population database 114, the rules database 116, and/or the external database 118. In some embodiments, the ML operation/training application 148 may include instruction for generating the trained fuzzy ruleset (e.g., trained fluid management rulesets 146) by training the fluid management ruleset (e.g., untrained fluid management rulesets 144) using the fuzzy machine learning model based on clinician expertise data (e.g., clinician expertise data stored on the external database 118) by approximating a continuous representation of a corresponding piecewise categorizing function using tropical geometry, as described in greater detail below with respect to FIG. 2, FIG. 3A, and FIG. 3B. Additionally, the ML operation/training application 148 may include instructions for generating the trained fuzzy ruleset or model based on at least the continuous representation of the corresponding piecewise categorizing function. In some embodiments, a user may review data from the ML operation/training application 148 and/or provide data to the ML operation/training application 148 via the input/output devices 122 and/or the input/output devices 172 of the user computing device 104. Moreover, the server computing device 102 may send data and/or information from the ML operation/training application 148 to the computing device 104 to be presented via the user interface 170, and the server computing device 102 may receive inputs provided by a user (e.g., input data for modifying and/or adjusting a model/ruleset) via the user interface 170 from the user computing device 104. In some embodiments, the server computing device 102 may send data and/or information from the ML operation/training application 148 to various components of the computing environment 100 (e.g., user computing devices 104, medical monitoring devices 108, medical pumps 110, additional medical devices 112, patient population database 114, rules database 116, and/or external database 118). For example, the server computing device 102 may send data and/or information from the ML operation/training application 148 to one or more medical devices of the computing environment 100 (e.g., medical monitoring devices 108, medical pumps 110, and/or additional medical devices 112). As another example, the server computing device 102 may send data and/or information from the ML operation/training application 148 to one or more databases/datastores of the computing environment 100 (e.g., patient population database 114, rules database 116, and/or external database 118) for subsequent storage.

In some embodiments, the memories 140 may have stored thereon additional modules and/or services for receiving and processing data from one or more other components of the environment 100 such as the medical monitoring device 108, the medical pump 110, the ML operation/training application 148, or the user computing device 104. For example, the memories 140 may store instructions for analyzing (e.g., using a trained ruleset, such as fluid management ruleset 146) clinical data (e.g., fluid balance data, vital signs data, etc.) from the medical monitoring device 108, the medical pump 110, and/or the additional medical device 112. Continuing with this example, the memories 140 may store instructions for outputting a fluid management recommendation for a patient generated based on applying (e.g., via the ML operation/training application 148) a trained ruleset to clinical data for the patient. As another example, the memories 140 may store instruction for controlling (e.g., command or control instruction) the medical pump 110 to, for example, administer a diuretics dosage to a patient based on a fluid management recommendation generated for the patient, as described below with respect to the ML operation/training application 148. As yet another example, the memories 140 may include instructions for receiving and implementing a trained fuzzy ruleset and/or a trained model (e.g., trained fuzzy ruleset 146, a trained fuzzy ruleset for fluid management) from the ML operation/training application 148.

In some embodiments, the fluid management module 142 and/or the ML operation/training application 148 may include additional computer-executable instructions corresponding to, for example, one or more of the operations represented by the flowcharts of this disclosure (e.g., the method 900 of FIG. 9). For example, the fluid management module 142 and/or the ML operation/training application 148 may include instructions for encoding clinician expertise data into fuzzy concepts and generating one or more untrained fluid management rulesets 144 based on the fuzzy concepts. Further, the fluid management module 142 and/or the ML operation/training application 148 may include instructions for initializing and training one or more machine learning models based on the one or more untrained fluid management rulesets 144 to generate the trained fluid management rulesets 146, or trained fuzzy rulesets, as discussed further below with respect to FIG. 2.

In some embodiments, the fluid management module 142 and/or the ML operation/training application 148 may include instructions for applying trained rulesets 146 (e.g., trained fuzzy rulesets) to clinical data (e.g., fluid balance data, respiratory data, oxygen saturation data, heart rate data, blood pressure data, body temperature data, etc.) for a patient to generate fluid management recommendation(s) (e.g., a recommendation to increase or decrease a diuretics dosage, a recommendation to increase or decrease a fluid dosage, etc.) for the patient. Additionally, the fluid management module 142 may send control/command instruction to the respective components of the environment 100 (e.g., the user computing device 104, the medical monitoring device 108, the medical pump 110, the additional medical device 112, the patient population database 114, the rules database 116, the external database 118, etc.), as well as any data relating thereto, generated thereby, or received via any communications interface(s) (e.g., communication interface 120, communication interface 160 of the user computing device 104, a communication interface of another component of the computing environment 100) and/or input device(s) (e.g., input/output device 122, the input/output device 172 of the user computing device 104, an input device of another component of the computing environment 100). For example, the fluid management module 142 may include instructions for sending data and/or information to the user computing device 104 to be displayed/presented to a user (e.g., via a graphical user interface) of the user computing device 104. As another example, the fluid management module 142 may include instructions for controlling, the medical pump 110, and/or the additional medical device 112. Expanding on this example, the fluid management module 142 may send instructions to the medical pump 110, that cause the medical pump 110 to increase or decrease a diuretics or fluid dosage for a patient (e.g., based on a fluid management recommendation generated for the patient). As yet another example, the fluid management module 142 may include instructions for receiving/obtaining information and/or data from the medical monitoring device 108 and/or the additional medical device 112. As still yet another example, the fluid management module 142 may include instructions for receiving/obtaining information and/or data from the patient population database 114, the rules database 116, and/or the external database 118.

Returning to the example user computing device 104 of FIG. 1, the user computing device 104 includes one or more communication interfaces 160, one or more user interfaces 170, one or more processors 180, and one or more memories 190. In some embodiments, the user computing device 104 may be included in and/or associated with medical environment. The user computing device 104 may be a computing device (e.g., a personal computer, a laptop, a smart phone, a tablet, a wearable device, etc.), or another suitable type of computing device or system (e.g., a collection of computing resources). In some embodiments, one or more user computing devices 104 may be included in the computing environment 100—for example, a user may access a first user computing device 104 that is a personal computer, a user may access a second user computing device 104 that is a smart phone, etc. Generally, a user of the user computing device 104 may be a clinician (e.g., a doctor, a physician, a surgeon, etc.) or another medical or healthcare professional (e.g., a nurse, a physician assistant, etc.). For ease of reading (and not limitation) purposes, the one or more user computing devices 104 may be referred to herein using the singular tense.

The one or more communication interfaces 160 may, similar to the communication interface 120, enable communication with other devices (e.g., server computing device 102) via, for example, the one or more networks 106. The example communication interface 160 may include any suitable type of communication interface(s) (e.g., wired and/or wireless interfaces such as a network interface controller) configured to operate in accordance with one or more suitable protocol(s). In some embodiments, the communication interface 160 may be a network interface controller (NIC) and may include any suitable NIC, such as wired/wireless controllers (e.g., Ethernet controllers), and facilitate bidirectional/multiplexed networking over the network 106 between the user computing device 104 and the server computing device 102 and/or other components of the environment 100 (e.g., medical monitoring device 108, medical pump 110, patient population database 114, rules database 116, external database 118, another computing device, etc.). In some embodiments, the communication interface 160 may include advanced features such as hardware acceleration, specialized networking protocols, etc.

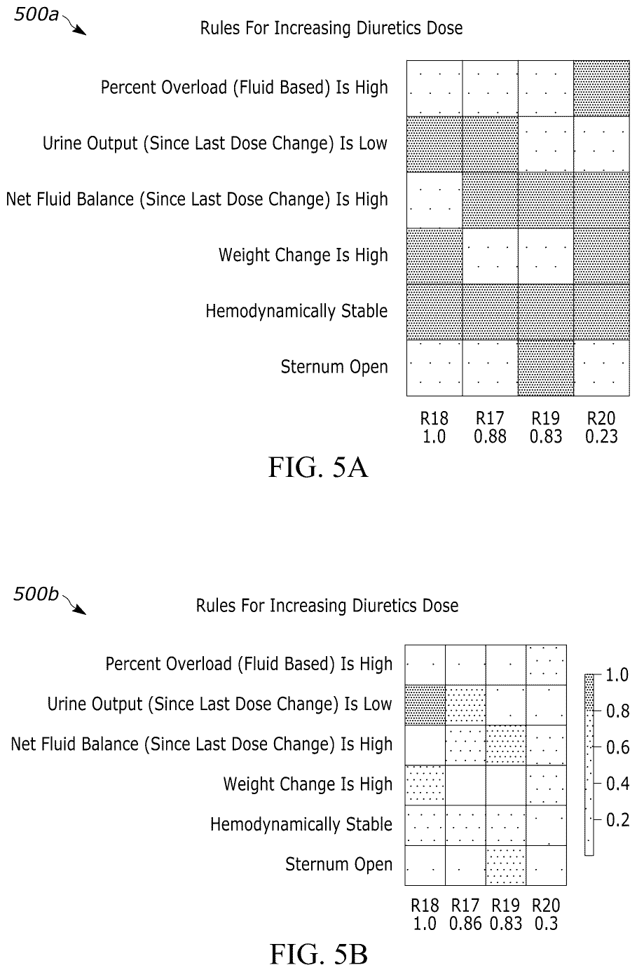

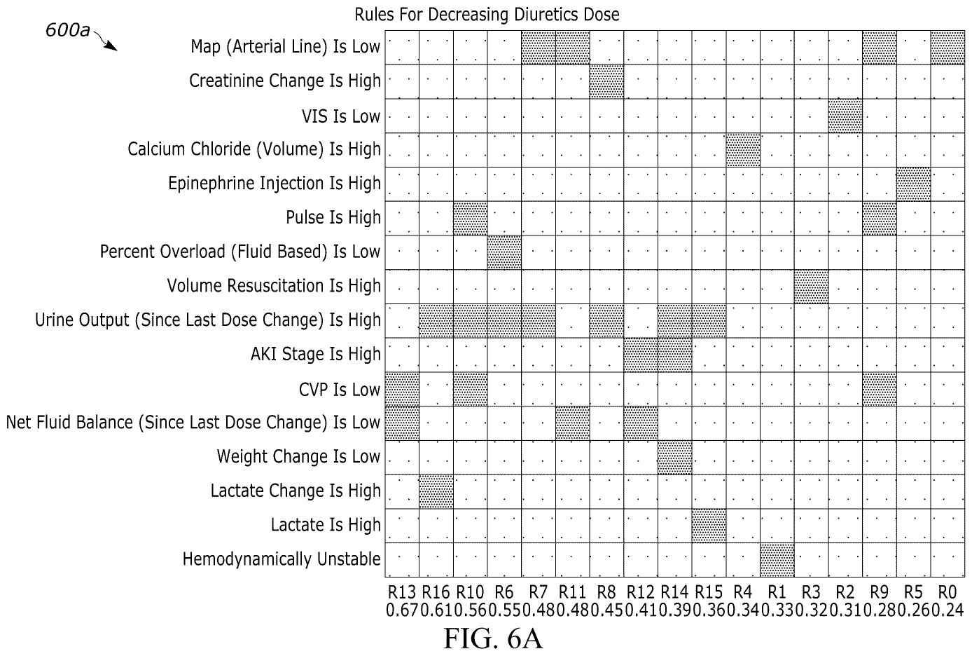

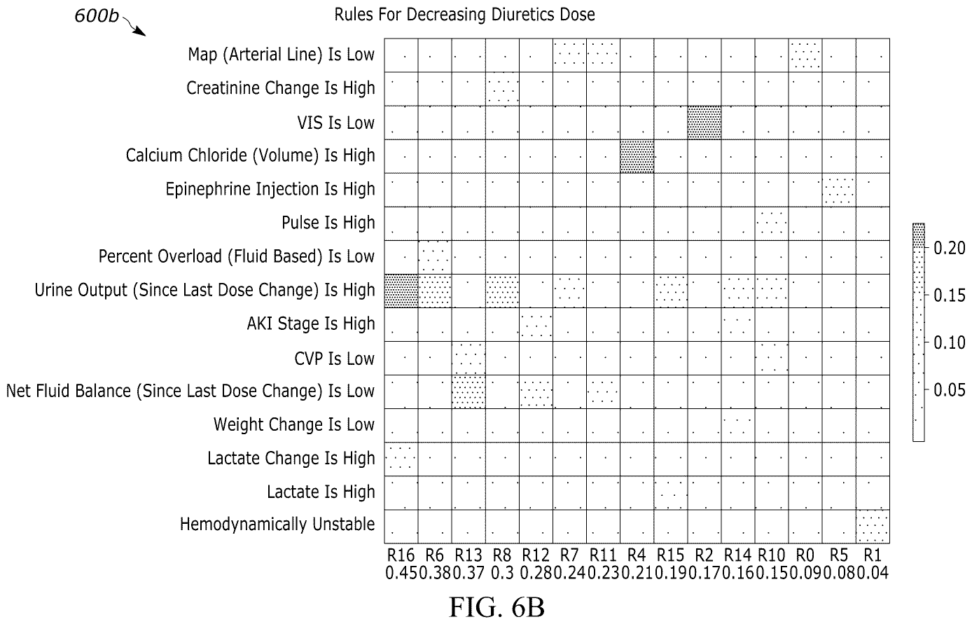

The one or more user interfaces 170 of the user computing device 104 may receive user input and communication of output data to the user. Further, the user interface 170 may be configured to present interactive graphical user interfaces (GUIs) to a user and receive feedback data from the user (e.g., via the input/output devices 172). For example, the user interface 170 may present interactive graphical user interfaces and/or graphical representations for a trained fluid management ruleset to a user, such as the graphical user interfaces depicted in FIG. 5A, FIG. 5B, FIG. 6A, or FIG. 6B, as discussed in greater detail below. As another example, a clinician may provide feedback data, such as an adjustment to a rule of a trained fluid management ruleset, using the user interface 170. Continuing with this example, the feedback data from the clinician may be sent to the server computing device 102 and the ML operation/training application 148 may update, change, modify, and/or retrain the model or ruleset based on the feedback data. The user interfaces 170 may include one or more input/output (I/O) device(s) 172. For instance, the I/O devices 172 may include one or more suitable types of user input devices, such as keyboards, touch screen displays, microphones, mice, touchpads, and/or any suitable types of remote and/or local user input devices. Similarly, the I/O devices 172 may include one or suitable types of output devices, such as touch screen displays, speakers, and the like. In some embodiments, the I/O devices 172 may include one or more display(s)/screen(s) 174 for presenting or displaying information to a user. The one or more displays/screens 174 may use any suitable display technology (e.g., LED, OLED, LCD, etc.). For example, the I/O devices 172 and/or the displays/screens 174 may be configured to present graphical representations of information and/or data associated with the embodiments described herein, such as information related to a trained ruleset or model, information relating to one or more particular rules of a ruleset, information related to training a ruleset or a model (e.g., information and/or data from the ML operation/training application 148). In some embodiments, the I/O device 172 and the display/screen 174 are integrated as a touchscreen display. In some embodiments, the user interfaces 170, the I/O devices 172, and/or the displays/screens 174 are not integral to the user computing device 104 and receive instructions from the user computing device 104 via wired and/or wireless transmissions over communication interface 160, for example. In some embodiments, the user interfaces 170, the I/O devices 172, and/or the displays/screens 174 may include one or more local interfaces, and/or may include one or more remote interfaces that are communicatively connected to the user computing device 104 via the network 106 (e.g., that are provided by an application, web browser, or other software executing on a device of a user). For ease of reading (and not limitation) purposes, the user interfaces 170, the I/O devices 172, and/or the displays/screens 174 may be referred to herein using the singular tense.

The processors 180 may include one or more microprocessors, controllers, and/or any suitable type of processor (e.g., one or more processors similar to the processors 130), and the memories 190 (e.g., one or more volatile memories, one or more non-volatile memories, or one or more non-transitory computer-readable media similar to the memories 140) may be accessible by the processor 180 (e.g., via a memory controller). The processor 180 may interact with the memory 190 to obtain, for example, machine-readable instructions and/or computer-executable instructions stored in the memory 190 corresponding to, for example, one or more of the operations represented by the flowcharts of this disclosure (e.g., the method 900 of FIG. 9). Additionally, the processors 180 may be communicatively coupled to and/or control the communication interface 160 to transmit and/or receive various information pursuant to executing of the instructions stored on the memories 190 and/or the memories 140 via, for example, the network 106.

As noted, the memories 190 may have stored thereon one or more modules, for example, as one or more sets of computer-executable instructions. In some aspects, the modules may include additional storage, such as one or more operating systems (e.g., Microsoft Windows, GNU/Linux, Mac OSX, etc.). The operating systems may be configured to run the modules during operation of the user computing device 104. For example, the modules may include additional modules and/or services for receiving and processing data from one or more other components of the environment 100 such as the server computing device 102, the medical monitoring device 108, the medical pump 110, or the rules database 116. The modules may be implemented using any suitable computer programming language(s) (e.g., Python, JavaScript, C, C++, Rust, C#, Swift, Java, Go, LISP, Ruby, Fortran, etc.). The modules may be configured to communicate with one another (e.g., via inter-process communication, via a bus, via sockets, pipes, message queues, etc.). In some embodiments, the modules may respond to network requests or other requests received via the network 106 (e.g., via the server computing device 102 or other components of the environment 100).

In operation, a clinician may be administering a fluid management procedure for a patient (e.g., a neonatal patient that has undergone congenital heart surgery). In an example scenario, the patient may be recovering from a surgery and the medical monitoring device 108 may collect clinical vital signs on a continuous basis throughout the recovery of the patient. Moreover, based on clinical data received from the medical monitoring device 108, a fluid management recommendation (e.g., a diuretics dosage recommendation or a fluid dosage recommendation) may be generated (e.g., via the ML operation/training application 148) by applying a trained fuzzy ruleset (e.g., a trained fuzzy ruleset such as trained ruleset 146) to the clinical data. Subsequently, the user computing device 104 may present (e.g., by the user interface 170) the fluid management recommendation and indications of corresponding rules to the clinician, via a graphical user interface such as the graphical user interface 500a of FIG. 5A and/or the graphical user interface 600a of FIG. 6A. In some embodiments, presenting the fluid management recommendation includes generating, and presenting to the patient, an alert including an indication of a request for the clinician to provide an adjustment to the fluid management recommendation. Additionally, the clinician may provide/input (e.g., via the user interface 170) feedback data including an adjustment to at least one rule of the trained fuzzy ruleset. Further, an adjusted fluid management recommendation may be generated by applying an updated fuzzy ruleset including the adjustment to the at least one rule to the clinical data, or subsequent clinical data (e.g., the most recent clinical data). In some embodiments, the adjusted fluid management recommendation and indications of corresponding rules included in the trained fuzzy ruleset may be presented to the clinician via a graphical user interface (e.g., graphical user interface 500a, graphical user interface 600a). Based on the fluid management recommendation or the adjusted fluid management recommendation, the medical pump 110 may deliver or administer a diuretics dosage or a fluid dosage to the patient.

Referring next to FIG. 2, an example application architecture 200 for the machine learning (ML) operation and training application 148 of the server computing device 102 discussed above with respect to FIG. 1 includes various modules to process data and train a machine learning model, such as encoding module 202, rules module 204, inference module 206, and/or model training module 208 (see Method 900, blocks 902-904). In some embodiments, the components of the ML operation/training application 148 may be communicatively connected to a database 210 via, for example, the communication interface 120 of the server computing device 102. Generally, the ML operation and training application 148 may train and/or apply a model using a tropical geometry-based interpretable machine learning method.

The ML operation/training application 148 may be capable of communicating with (e.g., via the communication interface 120 and over the network 106) components of the computing environment 100 and/or components of the application architecture 200, such as the one or more user computing devices 104, the patient population database 114, one or more medical devices, the rules database 116, and/or the external database 118. Additionally, the ML operation/training application 148 may include one or more communication interfaces that facilitate information flow between the components of the ML operation/training application 148 (e.g., between the encoding module 202 and the rules module 204, between the encoding module 202 and the rules module 204 and the model training module 208, etc.).

The database 210 can maintain a data structure such as a table of virtual user identifiers, corresponding user identifiers and/or characteristics, and cookies associated with the virtual user identifiers. The database can further maintain one or more data structures regarding database or user device identifiers and/or information, such as a tree, a linked list, a table, a string, or a combination thereof. In some implementations, the database 210 is part of or is included in the memories 140.

As mentioned above, the ML operation/training application 148 may include a number of logic modules, such as the encoding module 202, the rules module 204, the inference module 206, and/or the model training module 208. Depending on the implementation, each of the encoding module 202, rules module 204, inference module 206, and/or model training module 208 can be implemented as a software module, hardware module, or a combination of both. For example, each module can include a processing unit, server, virtual server, circuit, engine, agent, appliance, or other logic device such as programmable logic arrays configured to communicate with the database 210 and/or with other components of the computing environment 100 via the network 106. The ML operation/training application 148 may include instructions which cause the ML operation/training application 148 to perform operations discussed below with regard to any of and/or any combination of the encoding module 202, rules module 204, inference module 206, and/or model training module 208.

The encoding module 202 receives input data from the components of the computing environment 100. In particular, the encoding module 202 can receive input data from the database 210 and/or from any of the server computing device 102 (e.g., input data received via the input/output devices 122), the user computing device 104 (e.g., input data received via the input/output devices 172), the patient population database 114, one or more medical devices, the rules database 116, or the experimental database 118, via the network 106. The input data may be ordinal data, continuous data, categorical data, etc. Depending on the implementation, the input data can be raw data, such as ECG signals; processed data, such as survey data; fuzzy rules, such as a rule stating that if EF is low, and pVO2 is low, then evaluate for HT/MCS; or any other similarly desired input data.

In some implementations, the encoding module 202 then uses fuzzy theory to encode variables into multiple fuzzy sets. The encoding module 202 assigns a membership value in the range of [0,1] to each variable based on the observed value for a given fuzzy set, indicating the confidence of the variable belonging to a given concept and/or set (i.e., with 0 referring to no confidence and 1 referring to complete confidence). In some implementations, the encoding module 202 uses membership functions to calculate the membership values. In further implementations, the model training module 208 trains the membership functions while training the machine learning model as a whole.

The encoding module 202 determines concepts for the input variables. In particular, the encoding module 202 encodes the variables into humanly understandable fuzzy concepts. The fuzzy concepts approximate the concepts used by human experts during decision-making. For example, an expert may describe a metric used in making a determination as “low”, “medium”, or “high” without having a firm definition of what constitutes each concept. As examples, a clinician may describe a heart rate as low, medium, or high; a stock broker may describe a stock as being low, medium, or high; and a network security analyst may describe a level of activity in a network as low, medium, or high. Each expert may make the determination without having a firm bound on what constitutes each category, and may even have crossover between categories (e.g., a heart rate is “a little high”).

The encoding module 202 then sets trainable membership functions for each of the concepts. For example, in some implementations, the encoding module 202 defines three functions, l(x), m(x), and h(x). In such implementations, the membership functions are defined as

$$l ( x ) = {f}_{{ϵ}_{1}} ( \frac{{a}_{i , 2} - x}{{a}_{i , 2} - {a}_{i , 1}} ) - {f}_{{ϵ}_{1}} ( \frac{{a}_{i , 1} - x}{{a}_{i , 2} - {a}_{i , 1}} ) , h ( x ) = {f}_{{ϵ}_{1}} ( \frac{x - {a}_{i , 3}}{{a}_{i , 4} - {a}_{i , 3}} ) - {f}_{{ϵ}_{1}} ( \frac{x - {a}_{i , 4}}{{a}_{i , 4} - {a}_{i , 3}} ) , and m ( x ) = {f}_{{ϵ}_{1}} ( \frac{x - {a}_{i , 1}}{{a}_{i , 2} - {a}_{i , 1}} ) - {f}_{{ϵ}_{1}} ( \frac{x - {a}_{i , 2}}{{a}_{i , 2} - {a}_{i , 1}} ) - {f}_{{ϵ}_{1}} ( \frac{{a}_{i , 3} - x}{{a}_{i , 4} - {a}_{i , 3}} ) + {f}_{{ϵ}_{1}} ( \frac{{a}_{i , 4} - x}{{a}_{i , 4} - {a}_{i , 3}} ) - 1 , where {f}_{{ϵ}_{1}} ( x ) = {ϵ}_{1} log ( 1 + exp ( \frac{x}{{ϵ}_{1}} ) ) ,$$

tunable hyperparameters a_{i,1}<a_{i,2}<a_{i,3}<a_{i,4}, and 0<∈₁<1. In such implementations, a_{i,1}<a_{i,2}<a_{i,3}<a_{i,4} are trainable variables in the form of tunable hyperparameters. Depending on the implementation, the number of tunable hyperparameters varies dependent upon the number of categories considered. Although four tunable hyperparameters are described above, it will be understood that any number of hyperparameters may be used. For example, the tunable hyperparameters may include a_{i,1}<a_{i,2}< . . . <a_{i,w}<a_{i,w+1}. Similarly, the encoding module may calculate the membership functions according to the above using the tunable hyperparameters. Further, the membership functions have a smoothness modulated by ∈₁. Because

$${lim}_{{ϵ}_{1} \to 0} {f}_{{ϵ}_{1}} ( x ) = max ( 0 , x ) ,$$

when ∈₁ approaches 0, the membership functions approach trapezoidal or triangular membership functions.

As described above, the encoding module 202 then encodes the input variables xᵢ as membership values in the fuzzy concepts. It will be understood that though three concepts are described herein, the techniques as described herein may apply to any number of concepts, and three concepts are chosen for ease of illustration and explanation.

In some implementations, the encoding module 202 may determine whether to encode the variables into fuzzy concepts based on the type of data input received. For example, the encoding module 202 may determine to encode the input data variables into fuzzy concepts when the data input is an ordinal or continuous variable, but may instead directly encode categorical variables, such that x; is encoded into l₁(xⱼ), l₂ (xⱼ), . . . , l_{L}_{ⱼ} (xⱼ), where Lⱼ is the number of levels of xⱼ and only one of l₁ (xⱼ), l₂(xⱼ), . . . , l_{L}_{ⱼ} (xⱼ) has a value of 1, while all others have a value of 0.

The rules module 204 determines the most relevant concept from each variable for each rule and calculates a firing strength (e.g., a weight) for each rule. In some implementations, the architecture of the rules module 204 includes two layers. In such implementations, the first layer of the rules module 204 selects the most relevant concept from each variable for each rule, and the second layer calculates the rule firing strength for each rule. Although the rules module 204 is generally described with respect to two layers, it will be understood that the rules module 204 may have fewer or more layers, depending on the implementation.

In some implementations, the rules module 204 determines the most relevant concept from each variable with respect to each rule using an attention matrix A. A is the partitioned matrix formed by concatenating submatrices A₁, A₂, . . . , A_{H}, where Aₕ is the attention submatrix for the input variable xh and H=I+J is the total number of input variables. Further, I and J are the total number of ordinal/continuous variables and categorical variables, respectively. In some implementations, the rules module 204 treats ordinal and continuous variables differently from categorical variables. For example, for an ordinal and/or continuous variable xᵢ, the submatrix Aᵢ with entries A_{i,m,n} has dimension Cₚ×K, where Cₚ is the number of concepts for ordinal or continuous variables, and K is the number of rules utilized in the network. In some implementations, Cₚ=3 and refers to the concepts “high”, “medium”, and “low” as described with regard to the encoding module 202 above. Similarly, for a categorical variable xⱼ, the submatrix Aⱼ with entries A_{j,m,n} has dimension Lⱼ×K, where Lⱼ is the number of levels of xⱼ. As such, the attention matrix A has dimension (Cₚ·I+Σⱼ Lⱼ)×K.

In implementations in which Cₚ=3, the entry A_{i,1,k} in the attention matrix may represent the contribution of the ordinal or continuous variable xᵢ being “low” to rule k, the entry A_{i,2,k} in the attention matrix may represent the contribution of xᵢ being “medium” to rule k, and the A_{i,3,k} in the attention matrix may represent the contribution xᵢ being “high” to rule k. Depending on the implementation, the number may vary, and any A_{i,n,k} may correspond with the appropriate concept. In further implementations, entries in the attention matrix are all trainable and constrained to [0,1] by an activation function, such as a hyperbolic tangent activation function. A higher value in A indicates a higher contribution. For an input variable xᵢ, the corresponding output from the rules module 204, or the first layer of the rules module 204 in implementations in which the rules module 204 includes multiple layers, is \tilde{x}ᵢ, a vector of length K. Further, \tilde{x}_{i,k} is the kth element of \tilde{x}ᵢ and represents the firing strength of xᵢ involved in the kth rule. In implementations in which the rules module 204 treats ordinal and continuous variables differently from categorical variables, then \tilde{x}_{i,k}=A_{i,1,k}l (xᵢ)+A_{i,2,k}m(xᵢ)+ . . . +A_{i,n,k}h(xᵢ) for an ordinal or continuous variable xᵢ, and

$${\tilde{x}}_{j , k} = {\sum }_{d = 1}^{{L}_{j}} {A}_{j , d , k} {l}_{d} ( {x}_{j} )$$

for a categorical variable xⱼ.

In some implementations, the rules module 204 calculates rule firing strength by a connection matrix M of dimension H×K. In further implementations, a second layer of the rules module 204 separate from the first layer of the rules module 204 that selects the most relevant concept from each variable with respect to each rule. The rules module 204 constructs the kth rule as a combination of \tilde{x}_{1,k}, . . . , \tilde{x}_{H,k}. An entry M_{i,k} in the connection matrix M denotes the contribution of xᵢ to the kth rule. In some implementations, entries in the connection matrix are all trainable and constrained to [0,1] by an activation function, such as a hyperbolic tangent activation function, and a higher value indicates a higher contribution.

In order to calculate rₖ, the firing strength of the kth rule, the rules module 204 defines a parameterized T-norm,

$${T}_{{ϵ}_{2}} ( x , y ) = {g}_{{ϵ}_{2}}^{- 1} ( {g}_{{ϵ}_{2}} ( x ) + {g}_{{ϵ}_{2}} ( y ) ) with {g}_{{ϵ}_{2}}^{- 1}$$

as the inverse of g_{∈}_{₂}. For 0<∈₂<1, g_{∈}_{₂} in the range of [0, ∞) is defined as

$${g}_{{ϵ}_{2}} ( x ) = ( \frac{{ϵ}_{2}}{1 - {ϵ}_{2}} ) ( 1 - {x}^{\frac{{ϵ}_{2} - 1}{{ϵ}_{2}}} ) and {g}_{{ϵ}_{2}}^{- 1}$$

is defined as

$${g}_{{ϵ}_{2}}^{- 1} ( z ) = {( 1 - \frac{1 - {ϵ}_{2}}{{ϵ}_{2}} z )}^{\frac{{ϵ}_{2}}{{ϵ}_{2} - 1}} .$$

### Therefore,

$${T}_{{ϵ}_{2}} ( x , y ) = {( {x}^{\frac{{ϵ}_{2} - 1}{{ϵ}_{2}}} + {y}^{\frac{{ϵ}_{2} - 1}{{ϵ}_{2}}} - 1 )}^{\frac{{ϵ}_{2}}{{ϵ}_{2} - 1}}$$

with the behavior

$${lim}_{{ϵ}_{2} \to 1} {T}_{{ϵ}_{2}} ( x , y ) = xy and {lim}_{{ϵ}_{2} \to 0} ( x , y ) = min ( x , y ) .$$

Put another way, the rules module 204 can modulate the defined T-norm via ∈₂. In some implementations, the rules module 204 calculates the firing strength rₖ by applying the T-norm to multiple inputs. As such,

$${r}_{k} = {T}_{{ϵ}_{2}} ( {\tilde{x}}_{1 , k}^{{M}_{1 , k}} , {\tilde{x}}_{2 , k}^{{M}_{2 , k}} , … , {\tilde{x}}_{H , k}^{{M}_{H , k}} ) = {g}_{{ϵ}_{2}}^{- 1} ( {\sum }_{i = 1}^{H} {g}_{{ϵ}_{2}} ( {\tilde{x}}_{i , k}^{{M}_{i , k}} ) ) = {( {\sum }_{i = 1}^{H} {\tilde{x}}_{i , k}^{{M}_{i , k} \cdot ( \frac{{ϵ}_{2} - 1}{{ϵ}_{2}} )} - H + 1 )}^{\frac{{ϵ}_{2}}{{ϵ}_{2} - 1}} .$$

In some such implementations, the rules module 204 uses the learned entries in the connection matrix M to vary the contribution and/or weight of each input for a given rule within the T-norm calculation. Further, the lower value in M indicates a lower contribution to the rule firing strength. For example, for

$${\tilde{x}}_{1 , k}^{{M}_{1 , k}} ,$$

a lower M_{1,k} (e.g., closer to 0) means

$${\tilde{x}}_{1 , k}^{{M}_{1 , k}}$$

is closer to 1 and consequently contributes less to rₖ with the defined T-norm.

The inference module 206 classifies the variables based on the rule firing strength that the rules module 204 calculates. In some implementations, the inference module 206 classifies the variables into C classes. In further implementations, the inference module 206 includes C nodes, one for each class, that are fully connected to nodes of the rules module 204. The inference module 206 calculates the firing strength of each node o_{c} using the rule firing strengths rₖ with an inference matrix W of dimension K×C. An entry W_{j,c} denotes the contribution of the kth rule to the cth class. In some implementations, entries in the inference matrix are all trainable and positive. In further implementations, a higher value in the inference matrix indicates a higher contribution.

In some implementations, the inference module 206 defines a parametrized T-conorm to calculate o_{c}. In particular, the inference module 206 defines the parametrized T-conorm on two inputs as

$${Q}_{{ϵ}_{3}} ( x , y ) = {( {x}^{\frac{1}{{ϵ}_{3}}} + {y}^{\frac{1}{{ϵ}_{3}}} )}^{{ϵ}_{3}} ,$$

where 0<∈₃<1. The T-conorm has asymptotic behavior according to the following:

$${lim}_{{ϵ}_{3} \to 1} {Q}_{{ϵ}_{3}} ( x , y ) = x + y and {lim}_{{ϵ}_{3} \to 0} {Q}_{{ϵ}_{3}} ( x , y ) = max ( x , y ) .$$

Accordingly, the inference module 206 can modulate the defined T-conorm between addition and max by modifying ∈₃. In some such implementations, the inference module 206 then calculates o_{c} according to

$${o}_{c} = {Q}_{{ϵ}_{3}} ( {W}_{1 , c} {r}_{1} , {W}_{2 , c} {r}_{2} , … , {W}_{K , c} {r}_{K} ) = {( {\sum }_{k = 1}^{K} {( {W}_{k , c} {r}_{k} )}^{\frac{1}{{ϵ}_{3}}} )}^{{ϵ}_{3}} .$$

In some further such implementations, after the inference module 206 calculates o₁, o₂, . . . , o_{C}, the inference module 206 applies a softmax activation function to generate probabilities p₁, p₂, . . . , p_{C} of being in each class, which are all in [0,1] with

$${\sum }_{c = 1}^{C} {p}_{c} = 1 .$$

Because the softmax activation functions guarantees that

$${\sum }_{c = 1}^{C} {p}_{c} = 1 ,$$

the number of valid nodes in the inference module 206 can be set to C−1 to avoid ambiguity in rule representation. For example, when performing binary classification W_{:,0} can be set to 0 so that the model will only learn subspaces related to the positive class.

Further still in some implementations, the inference module 206 may produce a continuous output. More specifically, the inference layer for the regression TGFNN begins by defining a set of output membership functions associated with each rule. The regressed continuous output of the TGFNN generally retains the same structure for the input layer (e.g., where fuzzy concepts are generated) and the rule layer (e.g., where firing strengths for each fuzzy rule are computed) but may differ in the inference layer. In the inference layer, a weight matrix O learns weights that encode the relationship between rules and output variable concepts. Analogous to classification, as described above, which encodes the contributory role of each fuzzy rule to a given class via a trainable matrix, the regressed continuous output employs a rule-to-output weight matrix that learns parametric descriptors for a continuous variable. The network may infer a single continuous output value by employing modified membership functions and defuzzification. The TGFNN models scenarios in which the outcome of interest does not map neatly to discrete classes (e.g., “low,” “medium,” or “high” risk) but instead is better framed as a continuous variable (e.g., a predicted dosage quantity).

Each output concept is defined by a parameterized membership function having a component function

$${F}_{{ϵ}_{3}} ( y ) = {ϵ}_{3} log ( 1 + {e}^{\frac{y}{{ϵ}_{3}}} )$$

and membership function

$${z}_{k} = {F}_{{ϵ}_{3}} ( \frac{y - {a}_{1}}{{a}_{2} - {a}_{1}} ) - {F}_{{ϵ}_{3}} ( \frac{y - {a}_{2}}{{a}_{2} - {a}_{1}} ) + {F}_{{ϵ}_{3}} ( \frac{{b}_{2} - y}{{b}_{2} - {b}_{1}} ) - {F}_{{ϵ}_{3}} ( \frac{{b}_{1} - y}{{b}_{2} - {b}_{1}} ) - 1$$

where zₖ is the membership value of any output value y to the output membership function of rule rₖ. Each fuzzy rule rₖ is associated with its own membership function zₖ capturing how different potential output values y map to implied degrees of match for that rule's predicted outcome.

Each rule membership function zₖ is defined discretely as a vector of N incremental zₖ values along the entire range of y. The learned parameters of a₁, a₂, b₁, and b₂ define the shape and location of the membership function and are constrained such that a₁<a₂<b₁<b₂ so that the order of concepts is preserved. As such, the present techniques flexibly shift and scale the shape of the membership function so that it reflects domain-specific semantics (e.g., the range of plausible diuretic doses). The smoothness of the functions is controlled by the trainable parameter ∈₃. The location and shape of the membership functions can be predefined according to domain knowledge and the desired behavior of the network.

When performing inference, each membership function is scaled by the aggregate concept firing strength (e.g., calculated via the parameterized T-conorm described above for classification) according to \tilde{Z}ₖ=min (fₖ, Zₖ) which clips the top of the function. Each membership function zₖ is discretized over N points spanning the overall range of the continuous output variable. Consequently, for each n=1 . . . . N, the local membership Zₖ (yₙ) is computed. Because the TGFNN also computes each rule's firing strength fₖ from the preceding layer (e.g., via a T-norm), the network clips the membership function at a level determined by fₖ. Even if a particular rule's membership function Zₖ indicates a strong affinity for certain values of y, that affinity is reduced in proportion to the rule's overall firing strength ensuring that only rules firing strongly are weighted prominently in the final defuzzification.

After computing these clipped membership functions for all rules, the network aggregates them by taking the pointwise maximum across all rules such that

$${\tilde{Z}}_{agg} = {max}_{k} ( {\tilde{Z}}_{k} )$$

and the predicted value ŷ is defuzzified by computing the y value that lies at the centroid of \tilde{Z}_{agg}. The discrete centroid is calculated via

$$\hat{y} = \frac{{\sum }_{n = 1}^{N} {y}_{n} \cdot {\tilde{Z}}_{agg} ( {y}_{n} )}{{\sum }_{n = 1}^{N} {\tilde{Z}}_{agg} ( {y}_{n} )}$$

where n is the index of discrete values in y. By merging the contributions of multiple fuzzy rules, the system obtains a global fuzzy set \tilde{Z}_{agg} describing how likely each output value is for the given input conditions. To produce a single numeric prediction ŷ the TGFNN calculates the discrete centroid (e.g., the center of gravity) over those membership values.

Thus, the TGFNN with regression functionality defuzzifies the aggregated membership function into a single y value, ensuring a smooth, interpretable mapping from underlying fuzzy rules to a continuous result. Because all aspects from each fuzzy set's shape to the final inferred output are differentiable via the tropical geometry-based approximations, the network is trained end-to-end with gradient-based optimizers (e.g., Adam) for training the TGFNN to learn both the input-to-rule relationships and the rules' mapping onto continuous outputs (e.g., captured by the membership function parameters) in a unified process.

Domain knowledge and expertise may guide the initial positions of parameters a₁, a₂, b₁, and b₂, for example, if clinicians know the minimum and maximum practical bounds for a dosage. Over multiple epochs of training, the network refines these parameters in conjunction with the other layers (e.g., analogous to the classification training approach except that a mean squared error or mean absolute error loss replaces cross-entropy). By introducing these continuous membership functions and a centroid-based defuzzification, the TGFNN extends its interpretability advantage to real-valued predictions, allowing experts to trace how each rule and membership function contributed to the final numeric outcome.

Using the encoding module 202, the rules module 204, and the inference module 206, the ML operation/training application 148 is able to both extract and inject fuzzy rules. Put another way, the ML operation/training application 148 is able to extract and inject rules in a way that humans can understand. The entries in the attention matrix A and connection matrix M represent the contribution of individual concepts and individual variables to each rule. The entries in the inference matrix W gives the contribution of individual rules to each class.

In some implementations, the ML operation/training application 148 constructs a contribution matrix S using the attention and connection matrices A and M. The contribution matrix S expresses the contribution of individual concepts to each rule in the model. The matrix S is of the same dimension as attention matrix A. Put another way, the matrix S is a partition matrix formed by concatenating submatrices S₁, S₂, . . . , S_{H}. In some implementations, the ML operation/training application 148 treats ordinal and continuous variables different from categorical variables. In such implementations, for an ordinal or continuous variable xᵢ, the corresponding submatrix Sᵢ has dimension 3×K. Similarly, for a categorical variable xⱼ, Sⱼ has dimension Lⱼ×K. The ML operation/training application 148 calculates entries S_{i,d,k} of Sᵢ and S_{j,d,k} of Sⱼ as S_{i,d,k}=A_{i,d,k}×M_{i,k}, d∈{1,2,3} and S_{j,d,k}=A_{j,d,k}×M_{j,k}, d∈{1, 2, . . . , Lⱼ}, where k∈{1, 2, . . . , K}. The entry S_{i,d,k} is the contribution of the dth concept of xᵢ to the kth rule. S_{:,:,k} encodes the construction of the kth rule, while W_{k,:} captures the relationship between classes and the kth rule. Further, while the ML operation/training application 148 is described as calculating S_{i,d,k} using 3 fuzzy concepts and thereby limiting d∈{1,2,3}, it will be recognized that the ML operation/training application 148 can use any appropriate number of fuzzy concepts as described in detail above.

As an example of how the ML operation/training application 148 generates, modifies, and/or represents humanly understandable rules, the ML operation/training application 148 receives a dataset with four continuous input variables x₁, x₂, x₃, x₄ and determines a binary response (negative and positive). A, M, and W are trained and the ML operation/training application 148 can calculate S. Further, take entries S_{1,1,1}, S_{2,3,1}, S_{2,2,2}, and S_{3,1,2} of the contribution matrix S as close to 1, with all other entries of S close to 0. In the inference matrix W, W_{1,2} and W_{2,2} are close to 1 while W_{1,1} and W_{2,1} are close to 0. From the given S and W, the ML operation/training application 148 can summarize two rules from the trained network as follows: (1) If x₁ is low and x₂ is high, then the sample is positive; (2) If x₂ is medium and x₃ is low, then the sample is positive. Each rule is represented in (S_{;,;,1}, W_{1,;}) and (S_{;,;,2}, W_{2,:}), respectively. The ML operation/training application 148 can extract the definitions of low, medium, and high concepts from the parameters in the encoding module 202. The extracted rules mimic human logic and a user can use the extracted rules to justify the network decisions. In some implementations, the rules are not extracted, but instead the trained model is used as a neural network after being trained, as described in more detail below.

The model training module 208 trains the modules and algorithms described above (see Method 900, blocks 904). In some implementations, the model training module 208 trains the various modules and algorithms by back-propagation with an Adam optimizer. In further implementations, the model training module 208 trains the classification model using a calculated regular cross-entropy loss, loss_{ce}. In still further implementations, the model training module 208 adds an l₁ norm-based regularization term lossₗ_{₁} to the loss function to favor rules with a smaller number of concepts, which are more feasible to use in practice. Further, the model training module 208 calculates the correlation among encoded rules as a loss term loss_{corr} to avoid extracting redundant rules. The loss function is defined as:

$${loss}_{total} = {loss}_{ce} + {λ}_{1} {loss}_{{l}_{1}} + {λ}_{2} {loss}_{corr} ; {loss}_{{l}_{1}} = {‖ vec ( A ) ‖}_{1} + {‖ vec ( M ) ‖}_{1} ; and {loss}_{corr} = {\sum }_{i = 1}^{H - 1} {\sum }_{j = i + 1}^{H} vec ( {S}_{: , : , i} ) vec ( {S}_{: , : , i} ) ,$$

where λ₁ and λ₂ control the magnitude of the l₁ norm-based regularization term and correlation based regularization term, respectively. vec(⋅) denotes the vectorization of a matrix.

In some implementations, the ML operation/training application 148 constrains ∈₁, ∈₂, ∈₃ to be equal for simplicity. For example, the ML operation/training application 148 may initialize each of the three as 0.99 at the beginning of training and gradually reduce each of ∈₁, ∈₂, ∈₃ with the number of training steps. In some implementations, the scheduling of the ∈ values is defined as ∈=max (∈ₘᵢₙ, ∈·γ^{training}^{ₛ}^{teps}), where γ is the decay rate that can be tuned as a hyperparameter. ∈ₘᵢₙ is another hyperparameter, whose optimal value varies with different applications. In some implementations, the ML operation/training application 148 initializes ∈ to ∈=0.99 and reduces ∈ to improve model optimization as discussed in more detail below.

In some implementations, before model training, the model training module 208 randomly initializes training parameters (see Method 900, block 902). In further implementations, the model training module 208 may use practical rules (e.g., the untrained rulesets 144) from the data processing database 210, the server computing device 102, the rules database 116, and/or the experimental database 118 to improve and/or initialize the network parameters. For example, clinician expertise data associated with fluid management procedure for patients who have had heart surgery. Using such rules to initialize the parameters may improve performance, particularly when the size of the training dataset is small. In the example detailed above, take the generated rules as previously known rules within the rules database 116. In such an example, the model training module 208 could then initialize the matrices A, M, and W as: (1) A: A_{1,1,1}, A_{2,3,1}, A_{2,2,2}, A_{3,1,2} having a higher value and other entries in A_{:,:,1} and A_{:,:,2} having a lower value; (2) M: M_{1,1}, M_{2,1}, M_{2,2}, M_{3,2} having a higher value and other entries in M_{:,1} and M_{:,2} having a lower value; (3) W: W_{1,2}, W_{2,2} having a high value and W_{1,1}, W_{2,1} having a low value; and (4) other entries in A, M, and W being randomly initialized.

In some further implementations, the model training module 208 assesses and/or evaluates the machine learning model after each iteration of training. For example, the model training module 208 may calculate an accuracy, precision, recall, F₁ value (i.e., the harmonic mean of precision and recall), and/or the area under the receiver operating characteristic curve. In some such implementations, the model training module 208 calculates the evaluation metrics as follows:

$$accuracy = \frac{true positive + true negative}{all positives + all negatives} ; precision = \frac{true positives}{true positives + false positives} ; recall = \frac{true positives}{true positives + false negatives} ; and {F}_{1} = 2 ( \frac{recall * precision}{recall + precision} ) .$$

In further implementations, the model training module 208 calculates generalization gaps as the differences between metrics on validation and test sets. In such implementations, a higher generalization gap indicates greater overfitting.

Depending on the implementation, the model training module 208 outputs the trained model, which a user may use as a neural network to analyze input data. In some implementations, the model training module 208 outputs the trained model in addition to or in place of the trained ruleset. In implementations in which the model training module 208 outputs the trained model in place of the trained ruleset, the trained model may still use the trained ruleset to analyze input data and/or perform other functions as described herein.

In some implementations, the application architecture 200 performs the module functions as outlined above using one or more algorithms and/or a neural network. In further implementations, the application architecture 200 performs the module functions as outlined above to train one or more algorithms and/or a neural network. In some such implementations, to train the algorithms and/or neural network, the model training module 208 uses training data to improve the functionality of the modules and/or the models in question. In particular, in some implementations, the model training module 208 trains the algorithms and/or neural network using a supervised machine learning program or algorithm. The neural network may be a convolutional neural network, a deep learning neural network, or a combined learning module or program that learns in two or more features or feature datasets (e.g., determining the coordinates and classification for input data) in a particular area of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques. In some embodiments, the machine learning based algorithms may be included as a library or package executed on a computing platform (e.g., server computing device 102). For example, libraries may include the TENSORFLOW based library, the PYTORCH library, and/or the SCIKIT-LEARN Python library.

Machine learning may involve identifying and recognizing patterns in existing data (such as training a neural network based on labeled classes and training data) in order to facilitate making predictions or identification for subsequent data (such as using the neural network on new input data in order to generate new rulesets, improve old input rulesets, and/or condense rulesets).

Machine learning model(s) implemented on the neural network(s), such as the encoding module 202, rules module 204, and inference module 206 or the models created by the aforementioned modules, described herein for some embodiments, may be created and trained based upon example data (e.g., “training data” and related input data and/or input rules) inputs or data (which may be termed “features” and “labels”) in order to make valid and reliable predictions for new inputs, such as testing level or production level data or inputs. In supervised machine learning, a machine learning program operating as a neural network on a server, computing device, or otherwise processor(s), may be provided with example inputs (e.g., “features”) and their associated, or observed, outputs (e.g., “labels”) in order for the machine learning program or algorithm in the neural network to determine or discover rules, relationships, patterns, or otherwise machine learning “models” that map such inputs (e.g., “features”) to the outputs (e.g., “labels”), for example, by determining and/or assigning weights or other metrics to the model across its various feature categories. Such rules, relationships, or models may then be provided subsequent inputs in order for the neural network, executing on the server, computing device, or processor(s), to predict, based on the discovered rules, relationships, or models, an expected output.

In an example implementation, the ML operation/training application 148 may utilize a fuzzy machine learning model to refine and/or apply a fluid management ruleset specifically tailored for a population (e.g., a population of neonatal patients post-congenital heart surgery). By training the fuzzy model on historical medical data, including various fluid management procedures/treatments and their outcomes, the application 148 may generate a trained fuzzy ruleset for fluid management control. Further, such trained fuzzy rulesets may provide interpretable fluid management recommendations that can be generated for real-time or near-real-time clinical data (see Method 900, block 908).

In an example implementation, the application architecture 200 may be set up and implemented as follows. A 10-fold cross-validation may be used to evaluate model performance. For each iteration, the dataset can be split into training, validation, and test sets. A random search algorithm can be applied using the training set and validation set for hyperparameter tuning, including learning rate, batch size, λ₁, λ₂, and ∈ₘᵢₙ.

Compared to popular “black box” machine learning algorithms—such as random forest, SVM, and XGBoost—and other interpretable models—such as logistic regression, decision tree, and Explainable Boosting Machine (EBM)—evaluated using a 10-fold cross-validation, the techniques as described herein provide better accuracy, recall, precision, F1, and area under the ROC curve (AUC) than almost all such models while still providing transparency. Moreover, the techniques as described herein has significantly lower generalization error than such black box methods.

In an example implementation, the application architecture 200 receives eight input variables: x₁˜N(0,2), x₂˜N(5,3), x₃˜N(−1,5), x₄˜N(1,2), x₅˜N(−2,1), x₆˜Bernoulli(0,5), x₇˜N(0,1), and x₈˜N(0,1). Further, the following rules are true for the dataset. Should any of the rules apply to an observation, then the observation is positive and otherwise is negative: Rule A: x₂<3.8 and x₃>2 and x₆=1; Rule B: x₂>6.3 and x₃>2 and x₆=1; Rule C: x₁<1 and x₄>2 and x₆=0; Rule D: x₃>0 and x₅>1 and x₆=0; and Rule E: x₁>1 and x₅>1.5 and z₆=0. In some further example implementations, random noise sampled from N(0,0.01) is added to the input variables. Further, the application architecture 200 determines that the observations do not rely on x₇ and x₈, so the application architecture 200 deems the variables in question irrelevant.

In the above example, the application architecture 200 receives 400 samples from the dataset, the percentage of positive samples is 34.25%, and the percentages of samples with Rule A, Rule B, Rule C, Rule D, and Rule E are 8.25%, 7.50%, 9.00%, 10.75%, and 2.00%, respectively. The application architecture 200 generates a visual rule summary (e.g., graphical user interface 500a and/or graph 500b of FIG. 5A and FIG. 5B respectively) of a summarized ruleset generated using the input dataset. For example, the visual rule summary may indicate that Rule 1 corresponds with Rule C, Rule 2 corresponds with a union of Rule A and Rule B, Rule 3 corresponds with Rule D, and Rule 4 is similar to Rule E. As such, the application architecture 200 generates a majority of the rules accurately and generates the final rule similarly due to only 2.00% of samples being consistent with Rule E.

In another example implementation, the application architecture 200 receives nine input variables x₁˜N(0,2), x₂˜N(5,3), x₃˜N(−1,5), x₄˜N(1,2), x₅˜N (−2,1), x₆˜N (−1,4.4), x₇˜N(0,1.2), x₈˜N(0,1), and x₉˜N(0,1), and the sample is positive when

$$\frac{{( {x}_{1} + 0.5 {x}_{2} + {x}_{3} )}^{2}}{1 + {e}^{{x}_{6} + 2 {x}_{7}}} < 1.$$

In the example implementation, the application architecture 200 receives 400 samples from the dataset. A generated ruleset visualization may demonstrate that Rule 1 shows that “high” levels of x₆ and x₇ lead to a positive class, since 1+e^{x}^{₆}+2x₇ becomes larger, and thus more likely to cause the expression to be less than 1. Similarly, Rules 4 and 5 illustrate that a low x₃ and a high x₁ or a low/medium x₁ and medium x₃ can similarly lead to a positive class, as (x₁+0.5x₂+x₃)² becomes smaller.

Referring next to FIG. 3A, a diagram 300a illustrates methods for receiving an input dataset and extracting rules therefrom as well as for receiving a trained model and dataset and subsequently developing a summarized ruleset (see Method 900, blocks 902-906). The method 310 of FIG. 3A may be implemented in application architecture 200 as described with regard to FIG. 2 above. Though the method 310 is described below with regard to application architecture 200 and the computing environment 100, it will be recognized that any similarly suitable application and/or environment may be used to implement the method of FIG. 3A.

First, when extracting rules from data and/or training a ruleset using method 310, ML operation/training application 148 receives input data 312 from any of and/or any combination of the user computing device(s) 104, the input/output device 122 of the server computing device 102, the medical monitoring device 108, the medical pump 110, the additional medical device 112 the patient population database 114, the rules database 116, and/or the external database 118 (see Method 900, block 906). In some implementations, the input data comprises ordinal variables, continuous variables, categorical variables, crafted rules, or any other similar data as described herein. Depending on the implementation, the ML operation/training application 148 may receive the input data 312 directly at an encoding module 314, which may be the encoding module 202 as described with regard to FIG. 2 above.

The encoding module 314 then encodes membership values 316 for fuzzy concepts. In some implementations, the encoding module 314 assigns a membership value 316 in the range of [0,1] to each variable and/or component of the input data 312 based on the observed value for a given fuzzy set, indicating the confidence of the variable belonging to a given concept and/or set (i.e., with 0 referring to no confidence and 1 referring to complete confidence). In some implementations, the encoding module 314 uses membership functions to calculate the membership values 316. The encoding module 314 then transmits the membership values 316 to the rule module 318. In some implementations, the rule module 318 is the rules module 204 as described with regard to FIG. 2 above.

The rule module 318 then generates a ruleset and/or determines the most relevant concept from each variable for each rule. In some implementations, the rule module 318 further calculates a firing strength for each rule and/or a weight 320 for each generated rule. Put another way, the rule module 318 determines which rules of the ruleset have the greatest effect on the outcome. In some implementations, the rule module 318 determines the firing strength and/or weight 320 for each rule by a weighting system, such that each rule has potential to affect the outcome in accordance with the weight of the respective rule. In further implementations, the rule module 318 determines the firing strength and/or weight 320 for each rule by a priority system, such that the applicable rule with the greatest priority controls.

In some implementations, the rule module 318 generates a piecewise categorizing function based on the generated ruleset and/or the firing strength/weight for each rule. In particular, the piecewise categorizing function generally covers the generated ruleset and details the relationships between various input variables and the output according to the generated ruleset. After generating the piecewise categorizing function representative of the ruleset, the rule module 318 may approximate a continuous representation of the piecewise categorizing function using tropical geometry, such as a gradient descent function as described with more detail in regard to FIG. 9 below. In other implementations, the inference module 322 approximates the continuous representation rather than the rule module 318. The rule module 318 then transmits the ruleset, the piecewise categorizing function, the continuous approximation, the firing strength, and/or the weight 320 to the inference module 322. In some implementations, the inference module 322 is the inference module 206 as described with regard to FIG. 2 above.

The inference module 322 then classifies the variables based on the rule firing strength and/or weights 320 that the rule module 318 calculates. In some implementations, the inference module 322 further determines the firing strength of each class on the output using the rule firing strength and/or weights 320. In some implementations, the output 324 includes the classified variables and/or the class firing strength of the classes. In further implementations, the output 324 includes a trained model as described herein. In still further implementations, the output 324 is a basic model that a module, such as rule training module 208, trains.

The ML operation/training application 148 may also perform a rule summary method 330. As part of the rule summary method 330, the ML operation/training application 148 uses trained models 332, such as those described above with regard to output 324 and FIG. 2, and/or a dataset 334. In some implementations, the ML operation/training application 148 already has access to the trained models 332 and/or dataset 334, such as from the inference module 322 or from an internal database 210. In other implementations, the ML operation/training application 148 receives the trained models 332 and/or dataset 334 via the server computing device 102, the user computing device 104, the rules database 116, and/or the experimental database 118. Though FIG. 3A illustrates trained models, it will be understood that the ML operation/training application 148 may similarly perform the rule summary method 330 before or as part of training the models at the rule training module 208.

After receiving and/or retrieving the trained models 332 and/or dataset 334, the ML operation/training application 148 performs a distance matrix calculation 336. In some implementations, the ML operation/training application 148 performs the distance matrix calculation 336 by calculating weights for individual variables to individual rules. The ML operation/training application 148 then averages the calculated weights over all data samples in the dataset to construct a matrix A with a size in accordance with the number of variables and the number of rules. For example, the matrix A may have size n×m, where n is the number of variables and m is the number of rules. The ML operation/training application 148 then calculates the distance matrix based on the matrix A. In some implementations, the distance matrix is D=1−A^{T}A, where an entry d_{i,j} indicates the distance between rule i to rule j. In some implementations, the ML operation/training application 148 performs distance matrix calculation using the piecewise categorizing function representing the generated ruleset to generate a continuous representation of the piecewise categorizing function.

After performing the distance matrix calculation 336, the ML operation/training application 148 clusters 338 the rules into groups. In some implementations, the ML operation/training application 148 clusters 338 the rules using a hierarchical clustering technique. In further implementations, the hierarchical clustering technique uses an agglomerative or bottom-up approach, where each rule starts with a cluster and the ML operation/training application 148 successively merges similar clusters, minimizing the distance between pairs of clusters. In other implementations, the hierarchical clustering technique uses a divisive or top-down approach, where the rules start as a single cluster and the ML operation/training application 148 successively divides the cluster into similar, smaller clusters.

After clustering 338 the rules, the ML operation/training application 148 performs a representative rule selection 340. In some implementations, the ML operation/training application 148 selects representative rules from each cluster. In further implementations, after selecting the representative rules, the ML operation/training application 148 discards any remaining rules. In other implementations, the ML operation/training application 148 stores the remaining rules and/or a reference to the remaining rules in the database 210. Depending on the implementation, the ML operation/training application 148 may select the representative rules from each cluster based on the firing strength and/or weight 320 of each rule. For example, the ML operation/training application 148 may select the rule with the greatest weight 320 or contribution to the classification. In some implementations, the data processing system calculates the contribution and/or weight 320 of each rule as the average of the weights for a rule over data samples in the dataset. In further implementations, the ML operation/training application 148 performs or confirms representative rule selection 340 by iteratively determining a local minimum and/or maximum of the ruleset using a gradient descent algorithm and modifying the overall ruleset based on such. For example, depending on the implementation, the ML operation/training application 148 can remove any rules resulting in local minima or maxima and/or keeping only rules that result in local minima or maxima to construct a continuous approximation of the piecewise categorizing function representative of the ruleset. The ML operation/training application 148 then outputs the set of summarized rules 342 as summarized by the rule summary method 330.

Referring next to FIG. 3B, a diagram illustrates further systems and methods for receiving an input dataset and extracting rules therefrom. The system 300b may implement methods as described with regard to FIG. 9 below. Though the system 300b is described below with reference to the application architecture 200 and the computing environment 100, it will be recognized that any similarly suitable system may be used to implement FIG. 3B.

The diagram illustrates a system 300b including multiple modules and layers for analysis. In particular, the system 300b includes an input layer 350, encoding module 360, rule module 370, and inference module 380. In some implementations, the inference module 380 also serves as an output layer and may be referred to as such herein. In further implementations, the system 300b is part of application architecture 200 and may be the ML operation/training application 148. Similarly, each of the encoding module 360, rule module 370, and inference module 380 may include or be each of the encoding module 202, rules module 204, and inference module 206, respectively.

At the input layer 350, the system 300b receives input datasets. In some implementations, the input datasets include any of ordinal variables, continuous variables, categorical variables, rules, trained rules, and/or any combination thereof. In further implementations, the input layer identifies and separates ordinal and continuous variables 352 xᵢ from categorical variables 354 xⱼ. Though FIG. 3B illustrates two variables, it will be understood that this is for ease of illustration and understanding, and the system may receive any number of input variables divided in any proportion between ordinal and continuous variables 352, categorical variables 354, and/or other forms of input data.

The encoding module 360 receives the input data from the input layer 350 and encodes the input data into one or more fuzzy concepts. In some implementations, the encoding module 360 encodes the input data differently based on the variable type. For example, the encoding module 360 may encode ordinal and/or continuous variables 352 based on a predetermined number of fuzzy concepts as described with regard to FIG. 2. Similarly, the encoding module 360 may encode categorical variables 354 with regard to the number of layers L; each variable has, as described with regard to FIG. 2.

In the exemplary embodiment of FIG. 3B, the encoding module 360 encodes ordinal and/or continuous variables 352 into three fuzzy concepts, a low concept 362, a medium concept 364, and a high concept 366. In some implementations, the fuzzy concepts are the low concept 362

$$l ( {x}_{i} ) = {f}_{{ϵ}_{1}} ( \frac{{a}_{i , 2} - x}{{a}_{i , 2} - {a}_{i , 1}} ) - {f}_{{ϵ}_{1}} ( \frac{{a}_{i , 1} - x}{{a}_{i , 2} - {a}_{i , 1}} ) ,$$

the medium concept 364

$$m ( {x}_{i} ) = {f}_{{ϵ}_{1}} ( \frac{x - {a}_{i , 1}}{{a}_{i , 2} - {a}_{i , 1}} ) - {f}_{{ϵ}_{1}} ( \frac{x - {a}_{i , 2}}{{a}_{i , 2} - {a}_{i , 1}} ) - {f}_{{ϵ}_{1}} ( \frac{{a}_{i , 3} - x}{{a}_{i , 4} - {a}_{i , 3}} ) + {f}_{{ϵ}_{1}} ( \frac{{a}_{i , 4} - x}{{a}_{i , 4} - {a}_{i , 3}} ) - 1 ,$$

and the high concept 366

$$h ( {x}_{i} ) = {f}_{{ϵ}_{1}} ( \frac{x - {a}_{i , 3}}{{a}_{i , 4} - {a}_{i , 3}} ) - {f}_{{ϵ}_{1}} ( \frac{x - {a}_{i , 4}}{{a}_{i , 4} - {a}_{i , 3}} ) , where {f}_{{ϵ}_{1}} ( x ) = {ϵ}_{1} log ( 1 + exp ( \frac{x}{{ϵ}_{1}} ) ) ,$$

tunable hyperparameters a_{i,1}<a_{i,2}<a_{i,3}<a_{i,4}, and 0<∈₁<1 as defined above. In further implementations, the encoding module 360 encodes the categorical variables 354 into encoded concepts 368A-368L, defined as l₁(xⱼ), . . . , l_{L}_{ⱼ}(xⱼ), where only one of l₁(xⱼ), . . . , l_{L}_{ⱼ}(xⱼ) has a value of 1, while all others have a value of 0.

The rule module 370 determines a firing strength of each variable on each rule as well as the firing strength of each rule. In particular, the rule module determines a vector \tilde{x}ᵢ 372 comprising entries 372A-372K \tilde{x}_{i,1} to \tilde{x}_{i,K} for variable 352 xᵢ, as well as the corresponding vector 376 \tilde{x}ⱼ and entries 376A-376K \tilde{x}_{j,1} to \tilde{x}_{j,K} for variable 354 xⱼ. In some implementations, the rule module 370 determines the vectors and entries based on a determined attention matrix A and the corresponding entries for each concept and each entry of the vectors \tilde{x}ᵢ and \tilde{x}ⱼ. For example, the low concept 362 may represent the contribution of the variable xⱼ 352 on a first rule entry vector by the attention matrix entry A_{i,l,1} and on a Kth rule entry by the attention matrix entry A_{i,l,K}. Similarly, the encoded concept 368A may represent the contribution of the ordinal variable xⱼ 354 on a first rule entry by the attention matrix entry A_{j,1,1} and on a Kth rule entry by the attention matrix entry A_{j,1, K}. In some implementations, the rule module 370 uses the attention matrix entries to generate a piecewise categorizing function representative of the ruleset. For example, the rule module 370 may use the entries to determine for what fuzzy concepts a variable xᵢ contributes positively to the output and generate the piecewise categorizing function based on such.

The rule module 370 further determines the firing strength r₁, . . . , r_{K} 374A-374K based on a connection matrix M as described in more detail with regard to FIG. 2 above. An entry M_{i,k} in the connection matrix denotes the contribution of the variable to the kth rule, constructed from the rule entry vectors \tilde{x}_{1,k}, . . . , \tilde{x}_{H,k}. In particular, the firing strength of a rule K, r_{K} 374K, depends on the corresponding entry for each connection matrix, constructed for each variable. Put another way, M_{i,K}, M_{j,K}, and every other such matrix determines the firing strength r_{K} 374K as described with regard to FIG. 2 above.

The inference module 380 determines a firing strength for each class into which the system 300b classifies the variables. In particular, the inference module determines class firing strengths o₁, . . . , o_{C} 382A-382C. In some implementations, the inference module 380 determines the class firing strengths 382A-382C based on an inference matrix W. In some such implementations, each rule firing strength 374A-374K affects each class firing strength 382A-382C. For example, the rule firing strength r₁ 374A has a contribution on each class firing strength 382A-382C in accordance with the matrix entries W_{1,1}, . . . , W_{1,C}.

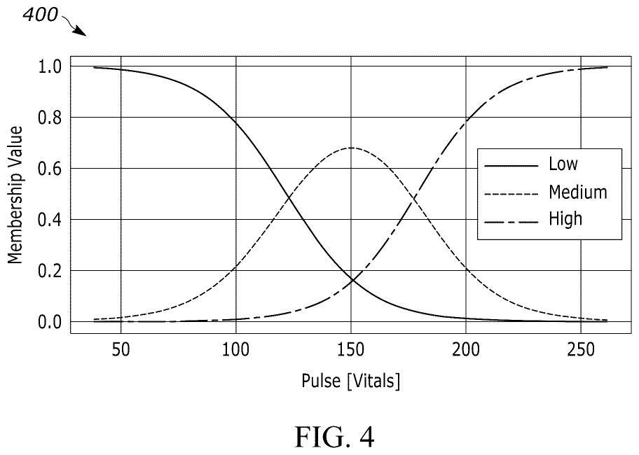

Referring next to FIG. 4, example graph 400 illustrates determined membership functions for concepts of an input variable involved in a rule. More particularly, the graph 400 illustrates the membership value for pulse, an input variable involved in a particular rule. Further, graph 400 illustrates that the membership value of pulse for the “low” concept is high when pulse is lower than approximately 125 beats per minute and the membership value of pulse for the “high” concept is high when pulse is higher than approximately 125 beats per minute and lower then approximately 175 beats per minute.

Referring next to FIG. 5A, example graphical user interfaces 500a illustrates an example visualization of a ruleset and concepts, as well as contributions to positive classes. More particularly, the example graphical user interfaces (GUI) 500a illustrates a visualization of rules for increasing a diuretics dose for an example patient (see Method 900, block 910). In the example GUI 500a, each column represents a rule for increasing a diuretics dosage and each row represents one “fuzzy” concept of a clinical feature with the exact values of each concept derived empirically during model training (e.g., using the ML operation/training application 148). Specifically, the example GUI 500a depicts four columns respectively corresponding to example rule 18 (R18), example rule 17 (R17), example rule 19 (R19), and example rule 20 (R20). The example GUI 500a includes six rows respectively corresponding to fuzzy concepts including fluid based percent overload, urine output, net fluid balance, weight change, hemodynamic stability, and whether the sternum is open. The number beneath each rule or column measures the importance of that rule to the model (e.g., R18 has a score of 1.0, R17 has a score of 0.88, R19 has a score of 0.83, R20 has a score of 0.23). For example, R18 for increasing the diuretics dose is: “If the urine output (since last dose change) is LOW and the weight change is HIGH and the patient IS hemodynamically stable, then increase the diuretics dose.” Additionally, some rules may be more important than other rules and certain fuzzy concepts may be more important than other fuzzy concepts. For example, the GUI 500a depicts that R18 is very important to the model with a score of 1.0 and within the rule, urine output is more important than percent overload and net fluid balance data.

Referring next to FIG. 5B, example graph 500b illustrates an example visualization of a ruleset and concepts, as well as contributions to positive classes. More particularly, the example graph 500b illustrates a visualization of the rules for increasing a diuretics dose for an example patient from FIG. 5A. Similarly, each column represents a rule for increasing a diuretics dosage (e.g., R18, R17, R19, R20) and each row represents one “fuzzy” concept of a clinical feature (e.g., fluid based percent overload, urine output, net fluid balance, weight change, hemodynamic stability, and whether the sternum is open). The color of each cell in the example graph 500b represents the relative importance of the individual concept within the rule using the scale shown to the right of the example graph 500b. For example, rule 18 for increasing the furosemide dose is: “If urine output is LOW and weight change is HIGH and the patient is hemodynamically stable, then increase the furosemide dose.” The rule is very important to the model with a score of 1.0 and within the rule, urine output is more important than weight change which is more important than hemodynamic stability.

Referring next to FIG. 6A, example graphical user interface 600a illustrates an example visualization of a ruleset and concepts, as well as contributions to positive classes. More particularly, the example graphical user interfaces (GUI) 600a illustrates a visualization of rules for decreasing a diuretics dose for an example patient. In the example GUI 600a, each column represents a rule for decreasing a diuretics dosage and each row represents one “fuzzy” concept of a clinical feature with the exact values of each concept derived empirically during model training (e.g., using the ML operation/training application 148).

Referring next to FIG. 6B, example graph 600b illustrates an example visualization of a ruleset and concepts, as well as contributions to positive classes. More particularly, the example graph 600b illustrates a visualization of the rules for decreasing a diuretics dose for an example patient from FIG. 6A. Similarly, each column represents a rule for decreasing a diuretics dosage and each row represents one “fuzzy” concept of a clinical feature. The color of each cell in the example graph 600b represents the relative importance of the individual concept within the rule using the scale shown to the right of the example graph 600b.

It should be understood that in some embodiments the example graphical user interfaces (e.g., the example GUI 500a of FIG. 5A and the example GUI 600a of FIG. 6A) described herein for presenting rules and/or rulesets to a user (e.g., a clinician) may include additional indicia, such as indicia indicative of the relative importance of particular fuzzy concepts (e.g., the gradient shading and key of FIG. 5B and FIG. 6B) and/or indicia indicative of the relative importance of particular fuzzy rules. Additionally or alternatively, the example graphical user interfaces may include further additional indicia, such as indicia indicative of: measures of uncertainty associated with each of the rules and/or model predictions, instances of changing importance of the rules and/or concepts as dictated by changes in the training data and/or context-specific modifications by end users, linkages between the rules and membership functions (e.g., to easily visualize the membership functions of the concepts comprising each rule), and/or situations when data may be missing such that a rule is not applicable. The GUI may present an explanation panel listing rules and contributions and provide quick-tweak controls (e.g., accept, edit, or reject the recommendation, or adjust salient rule weights) before issuing a command (see Method 900, block 912-914). Furthermore, the example graphical user interfaces and visualizations of rulesets/concepts discussed herein with respect to FIG. 5A, FIG. 5B, FIG. 6A, and FIG. 6B, may be presented and/or displayed by a user interface such as the user interface 170 of the user computing device 104.

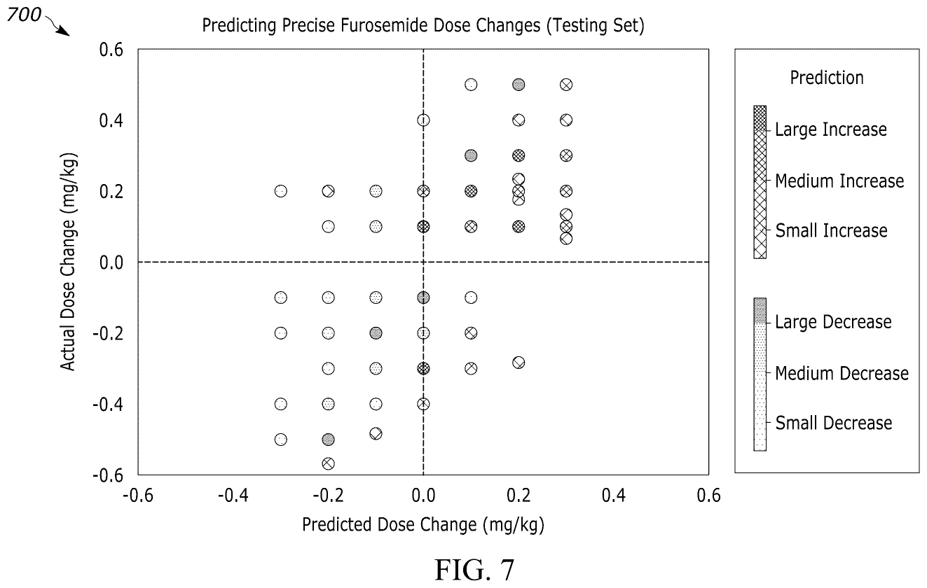

Referring next to FIG. 7, the example graph 700 illustrates a visualization of predicted and actual diuretics dose changes for a test set. In particular, the graph 700 is a plot of the predicted furosemide dose changes against the actual furosemide dose changes in the test set. It should be noted that the example model correctly predicted a furosemide increase or decrease 92.3% of the time with a mean absolute error of 0.11.

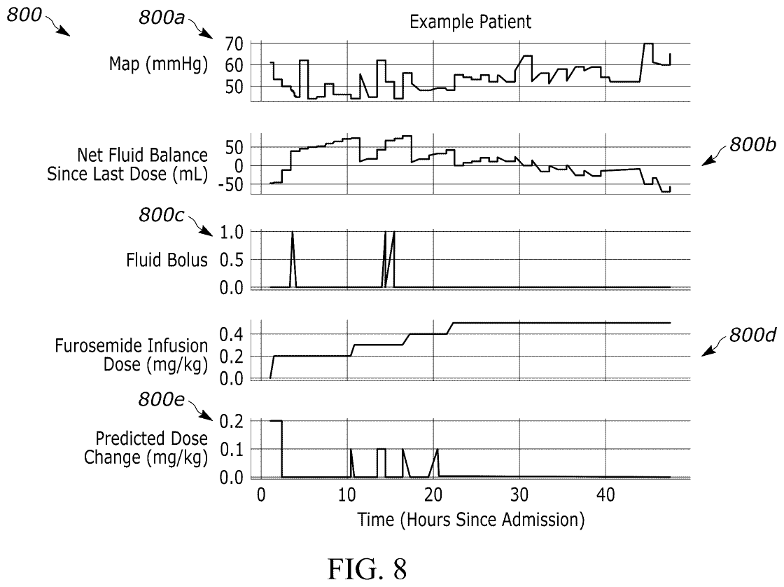

Referring next to FIG. 8, the example graphs 800a, 800b, and 800c illustrate example visualizations of three models used to inform a closed loop control system for fluid management of an example patient. In various embodiments, safety criteria include one or more guardrails (e.g., hemodynamic, respiratory, renal, or therapy-specific thresholds) that must be satisfied prior to action. Clinical variables are presented on the Y axis and time in hours since admission is presented on the X axis. More particularly, the example graphs 800a, 800b, and 800c illustrate visualizations of how each model could be used together to inform a closed loop control system. For example, a first model (e.g., the model corresponding to example graph 800a) could be used to predict an increase in furosemide during times of positive fluid balance and hemodynamic stability per its transparent rules. When the probability of predicting a furosemide increase breaches a pre-determined threshold, the first model output could be fed into a second model (e.g., the model corresponding to example graph 800b) to predict a specific dose increase. When “safety criteria” are met, such as low MAP, a third model (e.g., the model corresponding to example graph 800c) could provide a fluid bolus to help re-establish stability. Additionally, the example graph 800d illustrates the actual furosemide doses in this example patient and the example graph 800e illustrates the predicted furosemide doses in this example patient, for comparison.

Referring next to FIG. 9, a flowchart illustrates a method 900 for controlling fluid management for a patient. The method 900 of FIG. 9 may be implemented by the processors 130, the processors 180, and/or other suitable processors, etc., executing instructions stored on the memories 140, the memories 190, and/or another suitable non-transitory computer readable medium, etc., described above with respect to FIG. 1-9. Though the method below is described with regard to the computing environment 100, it will be recognized that any similarly suitable environment(s) and/or system(s) may be used to implement method 900.

At block 902, the machine learning (ML) operation and training application 148 may initialize a fuzzy machine learning model based on a fluid management ruleset for a population including the patient. In some embodiments, the population includes neonatal patients after congenital heart surgery. In some implementations, the ML operation/training application 148 obtains clinician expertise data associated with fluid management procedure for the population, encodes the clinician expertise data into fuzzy concepts, and generates the fluid management ruleset based on the fuzzy concepts.

At block 904, the ML operation/training application 148 may train the fuzzy machine learning model on historical medical data associated with the population to generate a trained fuzzy ruleset, the historical medical data including a plurality of indications of historical fluid management treatments for the population and associated treatment outcomes. In some embodiments, the ML operation/training application 148 trains, using a fuzzy machine learning model, the fluid management ruleset based on the clinician expertise data by: approximating, using tropical geometry, a continuous representation of the piecewise categorizing function, and generating, based on at least the continuous representation of the piecewise categorizing function, the trained fuzzy ruleset. In some embodiments, the historical medical data further includes electronic health records (EHR) data for a plurality of patients in the population and historical clinical data (e.g., vital signs) for a plurality of patients in the population.

At block 906, the ML operation/training application 148 may receive a clinical data for the patient (e.g., from the medical monitoring device 108). In some embodiments, the ML operation/training application 148 and/or the fluid management module 142 obtains clinical data for the patient on a continuous basis, wherein fluid management recommendations are generated based on the most recent clinical data and on a periodic basis. In some embodiments, the clinical data for the patient include one or more of: fluid balance data, respiratory data, oxygen saturation data, heart rate data, blood pressure data, or body temperature data.

At block 908, the ML operation/training application 148 and/or the fluid management module 142 may generate a fluid management recommendation by applying the trained fuzzy ruleset to the clinical data for the patient. In some embodiments, the fluid management recommendation is a diuretics dosage recommendation or a fluid dosage recommendation.

At block 910, the user interface 170 (e.g., via a graphical user interface such as the graphical user interface 500a of FIG. 5A or the graphical user interface 600a of FIG. 6A) may present the fluid management recommendation and one or more first indications of corresponding rules included in the trained fuzzy ruleset to a clinician. In some embodiments, presenting the fluid management recommendation comprises: generating an alert including an indication of a request for the clinician to provide an adjustment to the fluid management recommendation; and presenting, via the user interface 170 and/or a graphical user interface, the alert to the clinician. In further embodiments, the user interface 170 may present an explanation panel listing rules and their contributions and provide accept or reject controls prior to issuing a command.

At block 912, the ML operation/training application 148 and/or the fluid management module 142 may receive (e.g., via the user interface 170) feedback data input by a clinician including an adjustment to at least one rule of the trained fuzzy ruleset.

At block 914, the ML operation/training application 148 and/or the fluid management module 142 may generate an adjusted fluid management recommendation by applying an updated fuzzy ruleset to the clinical data.

At block 916, the ML operation/training application 148 and/or the fluid management module 142 may present (e.g., via the user interface 170) the adjusted fluid management recommendation and one or more second indications of corresponding rules included in the trained fuzzy ruleset to the clinician.

Additionally, the method 900 may include administering, via a medical pump (e.g., the medical pump 110), a diuretics dosage or a fluid dosage to the patient based on the fluid management recommendation or the adjusted fluid management recommendation.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive, and should instead be understood as potentially combinable if such combinations are permissive in any way. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

It will be appreciated that some embodiments may be comprised of one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (e.g., comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

Moreover, the patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

1. A computer-implemented method for controlling fluid management for a patient, the computer-implemented method comprising:
initializing, by one or more processors, a fuzzy machine learning model based on a fluid management ruleset for a population including the patient;
training, by the one or more processors, the fuzzy machine learning model on historical medical data associated with the population to generate a trained fuzzy ruleset, the historical medical data including a plurality of indications of historical fluid management treatments for the population and associated treatment outcomes;
receiving, by the one or more processors, clinical data for the patient;
generating, by the one or more processors, a fluid management recommendation by applying the trained fuzzy ruleset to the clinical data for the patient;
presenting, by the one or more processors and via a graphical user interface, the fluid management recommendation and one or more first indications of corresponding rules included in the trained fuzzy ruleset to a clinician;
receiving, by the one or more processors and via the graphical user interface, feedback data input by a clinician including an adjustment to at least one rule of the trained fuzzy ruleset;
generating, by the one or more processors, an adjusted fluid management recommendation by applying an updated fuzzy ruleset to the clinical data; and
presenting, by the one or more processors and via the graphical user interface, the adjusted fluid management recommendation and one or more second indications of corresponding rules included in the trained fuzzy ruleset to the clinician.

2. The computer-implemented method of claim 1, wherein the population includes neonatal patients after congenital heart surgery.

3. The computer-implemented method of claim 1, further comprising:
obtaining, by one or more processors, clinician expertise data associated with fluid management procedure for the population;
encoding, by the one or more processors, the clinician expertise data into fuzzy concepts; and
generating, by the one or more processors, the fluid management ruleset based on the fuzzy concepts.

4. The computer-implemented method of claim 3, wherein generating the trained fuzzy ruleset comprises:
training, by the one or more processors and using the fuzzy machine learning model, the fluid management ruleset based on the clinician expertise data by:
approximating, using tropical geometry, a continuous representation of a piecewise categorizing function, and
generating, based on at least the continuous representation of the piecewise categorizing function, the trained fuzzy ruleset.

5. The computer-implemented method of claim 1, wherein the fluid management recommendation is a diuretics dosage recommendation or a fluid dosage recommendation.

6. The computer-implemented method of claim 5, further comprising:
administering, by the one or more processors and via a medical pump device, a diuretics dosage or a fluid dosage to the patient based on the fluid management recommendation or the adjusted fluid management recommendation.

7. The computer-implemented method of claim 1, wherein the historical medical data further includes electronic health records (EHR) data for a plurality of patients in the population and historical clinical data for a plurality of patients in the population.

8. The computer-implemented method of claim 1, further comprising:
obtaining, by the one or more processors, clinical data for the patient on a continuous basis, wherein fluid management recommendations are generated based on most recent clinical data and on a periodic basis.

9. The computer-implemented method of claim 8, wherein receiving the feedback data comprises:
generating, by the one or more processors, an alert including an indication of a request for the clinician to provide an adjustment to the fluid management recommendation; and
presenting, via the graphical user interface, the alert to the clinician.

10. The computer-implemented method of claim 8, wherein the clinical data for the patient include one or more of: fluid balance data, respiratory data, oxygen saturation data, heart rate data, blood pressure data, or body temperature data.

11. A computing system for controlling fluid management for a patient, comprising:
one or more medical devices including at least a medical monitoring device and a medical pump device;
one or more processors;
one or more memories including computer-executable instructions that, when executed by the one or more processors, cause the computing system to:
initialize a fuzzy machine learning model based on a fluid management ruleset for a population including the patient;
train the fuzzy machine learning model on historical medical data associated with the population to generate a trained fuzzy ruleset, the historical medical data including a plurality of indications of historical fluid management treatments for the population and associated treatment outcomes;
receive, from the medical monitoring device, clinical data for the patient;
generate a fluid management recommendation by applying the trained fuzzy ruleset to the clinical data for the patient;
presenting, via a graphical user interface, the fluid management recommendation and one or more first indications of corresponding rules included in the trained fuzzy ruleset to a clinician;
receiving, via the graphical user interface, feedback data input by a clinician including an adjustment to at least one rule of the trained fuzzy ruleset;
generate an adjusted fluid management recommendation by applying an updated fuzzy ruleset to the clinical data; and
present, by the one or more processors and via the graphical user interface, the adjusted fluid management recommendation and one or more second indications of corresponding rules included in the trained fuzzy ruleset to the clinician.

12. The computing system of claim 11, wherein the population includes neonatal patients after congenital heart surgery.

13. The computing system of claim 11, the one or more memories further including computer-executable instructions that, when executed by the one or more processors, cause the computing system to:
obtain clinician expertise data associated with fluid management procedure for the population;
encode the clinician expertise data into fuzzy concepts; and
generate the fluid management ruleset based on the fuzzy concepts.

14. The computing system of claim 13, the one or more memories further including computer-executable instructions that, when executed by the one or more processors, generate the trained fuzzy ruleset by causing the computing system to:
train, using the fuzzy machine learning model, the fluid management ruleset based on the clinician expertise data by:
approximating, using tropical geometry, a continuous representation of a piecewise categorizing function, and
generating, based on at least the continuous representation of the piecewise categorizing function, the trained fuzzy ruleset.

15. The computing system of claim 11, wherein the fluid management recommendation is a diuretics dosage recommendation or a fluid dosage recommendation.

16. The computing system of claim 15, the one or more memories further including computer-executable instructions that, when executed by the one or more processors, cause the computing system to:
administer, via the medical pump device, a diuretics dosage or a fluid dosage to the patient based on the fluid management recommendation or the adjusted fluid management recommendation.

17. The computing system of claim 11, wherein the historical medical data further includes electronic health records (EHR) data for a plurality of patients in the population and historical clinical data for a plurality of patients in the population.

18. The computing system of claim 11, the one or more memories further including computer-executable instructions that, when executed by the one or more processors, cause the computing system to:
obtain clinical data for the patient on a continuous basis, wherein fluid management recommendations are generated based on most recent clinical data and on a periodic basis.

19. The computing system of claim 18, the one or more memories further including computer-executable instructions that, when executed by the one or more processors, receive the feedback data by causing the computing system to:
generate, by the one or more processors, an alert including an indication of a request for the clinician to provide an adjustment to the fluid management recommendation; and
present, via the graphical user interface, the alert to the clinician.

20. The computing system of claim 18, wherein the clinical data for the patient include one or more of: fluid balance data, respiratory data, oxygen saturation data, heart rate data, blood pressure data, or body temperature data.
